(19) United States
(12) Patent Application Publication
(10) Pub. No.: US 2023/0027004 A1
(43) Pub. Date: Jan. 26, 2023

(54) CLOSED-SYSTEM AND METHOD FOR AUTOLOGOUS AND ALLOGENEIC CELL THERAPY MANUFACTURING

(71) Applicant: Kite Pharma, Inc., Santa Monica, CA (US)

(72) Inventors: Nathaniel W. Freund, Los Angeles, CA (US); Kaiyuan Jiang, Los Angeles, CA (US); Suchit Sahai, West Hills, CA (US); Hsing-Chuan Tsai, Santa Monica, CA (US); Maolu Li, Hacienda Heights, CA (US); Joshua Ray Plat, Ventura, CA (US); Waleed Haso, Santa Monica, CA (US); Nitin Agarwal, Santa Monica, CA (US); Qi Cai, Champaign, IL (US); Luis Diaz, Newbury Park, CA (US); Kent S. Young, Los Angeles, CA (US)

(21) Appl. No.: 17/852,918

(22) Filed: Jun. 29, 2022

Related U.S. Application Data
(60) Provisional application No. 63/217,378, filed on Jul. 1, 2021.

(51) Int. Cl. C12N 5/0783; C12N 15/86; C12N 15/87

(52) U.S. Cl. CPC C12N 5/0636; C12N 15/86; C12N 15/87; C12N 2740/15043

(57) ABSTRACT

A system and method for manufacturing engineered human lymphocytes for cell therapies, including isolating targeted cells of interest from apheresis starting material using an acoustic separation device and activating the targeted cells of interest in situ with, in certain aspects, antibody-coated surface in an enclosed vessel. Also, the method includes transfecting the targeted cells of interest with construct-encoded lentiviral vectors, retroviral vectors, adeno-associated vectors or non-viral vectors in the enclosed vessel. The cells of interest may then be transfected with viral or non-viral genetic material using an electroporation device. Transfected cells may then be expanded to a desired dose using an expansion feeding method. Also, the method may include combining the targeted cells of interest with cryoprotectant reagents and buffers to create a final formulation.

### CROSS-REFERENCES TO RELATED APPLICATIONS

This application claims the benefit of priority to U.S. Provisional Patent Application No. 63/217,378, filed 1 Jul. 2021 and titled “Closed-System and Method for Autologous and Allogenic Cell Therapy Manufacturing,” the entirety of which is incorporated by reference herein.

### BACKGROUND

In recent years, advances in medical technology have led to the emerging use of immunotherapies to treat different types of illnesses and diseases, including various forms of cancer. Generally, immunotherapy is the treatment of disease by stimulating or suppressing an immune response. Often, modified versions of a patient's own biological material, such as immune cells, are reintroduced into the patient in order to initiate and/or supplement the immune response.

For example, engineered immune cells have been shown to possess desired qualities in therapeutic treatments, particularly in oncology. Two main types of engineered immune cells are those that contain chimeric antigen receptors (termed “CARs” or “CAR-Ts”) and T-cell receptors (“TCRs”). These engineered cells are engineered to endow them with antigen specificity while retaining or enhancing their ability to recognize and kill a target cell. Chimeric antigen receptors may comprise, for example, (i) an antigen-specific component (“antigen binding molecule”), (ii) an extracellular domain, (iii) one or more costimulatory domains, and (iv) one or more activating domains. Each domain may be heterogeneous, that is, comprised of sequences derived from (or corresponding to) different protein chains.

Because many patients that undergo immunotherapy are critically ill, a crucial factor in the efficacy of such immunotherapy procedures is the ability to provide the modified biological material to the patient as quickly as possible and in a safe clean and uncontaminated manner.

What is needed is a system and method for manufacturing engineered human lymphocytes where all or a combination of unit operations may be completed in an automated closed-system approach designed to create modular or continuous scale-down/scale-out unit operations. Furthermore, what is needed is a method that may consolidate raw materials and single-use kits, reduce labor and clean-room environmental monitoring costs associated with quality control, quality assurance, sterility, and process deviations. Also, what is needed is a system and method that allows for consolidation of process equipment and unit operations enabled that allows for economies of scale and scale-up/scale-out to increase run rates, suite capacity, and reduce lot release time.

### SUMMARY

Briefly, and in general terms, the present disclosure is directed to a system and method for manufacturing engineered human lymphocytes for cell therapies, including isolating targeted cells of interest from apheresis starting material using an acoustic separation device and activating the targeted cells of interest in situ with antibody-coated surface in an enclosed vessel. Also, the method includes transducing the targeted cells of interest with construct-encoded lentiviral vectors, retroviral vectors or adeno-associated vectors in the enclosed vessel. The cells of interest may then be transfected with genetic or non-genetic material using an electroporation device. Transfected cells are then expanded to a desired dose using an expansion feeding method. Also, the method may include combining the targeted cells of interest with cryoprotectant reagents and buffers to create a final formulation.

In certain embodiment, the method includes isolating targeted cells via positive or negative selection using density gradient, magnetic bead, or acoustic forces. Furthermore, the targeted cells of interest may include markers for CD3, CD4, CD8, CD14, CD19, CD25, CD27, CD28, CD56, CD69, CD95, CCR7, CD62L, CD45RA/RO, PD1, OX40, ICOS expression or a combination.

In one embodiment, activation of the targeted cells may occur sequentially with isolating using antibody conjugated or physically coated beads, labels, surfaces, or particles bound to target cells. In other embodiments, the method may include activating the targeted cells concurrently with isolating using antibody conjugated beads, labels, or particles bound to target cells.

In one embodiment, the method includes washing the activated target cells with centrifugation or buffer exchange. The method may also include conducting a preliminary wash of the apheresis starting material. Also, the method may include labelling targeted cells using antibody conjugated beads, labels or particles.

In one embodiment, the targeted cells and conjugated beads, labels, or particles are combined under static conditions, rocking conditions, or circulating conditions.

The apheresis starting material may be between 150 mL and 300 mL in certain embodiments. Furthermore, the at-scale activation culture volume range may be from 100 mL to 6 L. The method may occur under various conduction, and in one embodiment, activating the targeted cells of interest occurs up to 96 hours at 37° C. and 5% CO2.

In one embodiment, the method may include performing a closed-system centrifugation wash to the target cells of interest after activating the target cells of interest.

In yet another embodiment, the method may include transducing the targeted cells of interest with construct-encoded lentiviral vectors, retroviral vectors or adeno-associated vectors occurs using an enhancing reagent. Furthermore, the enhancing reagent is retronectin, protamine sulfate, polybrene, vectofusin-1, Sirion AdenoBOOST™, Sirion LentiBOOST. In another embodiment, the method includes transducing the targeted cells of interest with construct-encoded lentiviral vectors, retroviral vectors or adeno-associated vectors occurs without the use of an enhancing reagent. The conductions for transducing the targeted cells of interest with construct-encoded lentiviral vectors, retroviral vectors or adeno-associated vectors may occur for 1 to 72 hours at a temperature between 15° C. to 37° C.

In one embodiment, activating the target cells of interest occurs sequentially with transducing the target cells of interest. In another embodiment, activating the target cells of interest occurs concurrently with transducing the target cells of interest.

In another embodiment, the present disclosure is directed to a system and method for manufacturing engineered human lymphocytes for cell therapies. In this embodiment, the method includes isolating targeted cells of interest from apheresis of a healthy donor using an acoustic separation device and activating the targeted cells of interest by stimulation of antibody receptors in the presence of IL-2 for 24 to 96 hours in an enclosed vessel. The method may include washing activated targeted cells of interest using a buffer exchange module. Following the wash, the method includes transfecting the targeted cells with genetic or non-genetic material using an electroporation device and transducing the targeted cells of interest after transfection with construct-encoded lentiviral vectors or retroviral vectors with retronectin in the enclosed vessel. The method may also include expanding the targeted cells of interest after transduction to a desired dose and then depleting the targeted cells of interest after expansion using a negative selection stepwise isolation step to deplete the unedited TCRab+ cells. After depletion, the method may include combining the targeted cells of interest with cryoprotectant reagents and buffers to create a final formulation.

In certain embodiments, the targeted cells of interest include markers for CD3, CD4, CD8, CD14, CD19, CD25, CD27, CD28, CD56, CD69, CD95, CCR7, CD62L, CD45RA/RO, PD1, OX40, ICOS expression or a combination. Also, the method may include washing activated targeted cells of interest using a buffer exchange to concentrate the cells between 15-100 e6 cells/mL in media. The buffer exchange processes at least 2.5 E9 cells per hour and 0.1 to 10 E9 cells per lot in one embodiment. The method may also include washing the targeted cells of interest after expansion and before depletion using acoustic separation to achieve a cell concentration of 50-300 e9 cells/mL in 200-500 mL. In one embodiment, the method includes suspending the targeted cells of interest after depletion in a cryopreservation media.

The following aspects are exemplary of the disclosure and non-limiting:

1. A method for manufacturing CAR expressing human lymphocytes, comprising:
(a) isolating target cells from donor sourced starting material using an isolation technique selected from the group consisting of acoustic separation, antibody-conjugated magnetic beads, density gradient separation, magnetic levitation, antibody conjugated labels, microspheres and any combination thereof;
(b) optionally, contacting the target cells with an activating molecule;
(c) transducing the target cells with CAR construct-encoded lentiviral vectors, retroviral vectors or adeno-associated vectors in an enclosed vessel, a fluidic channel and any combination thereof; and
(d) transfecting the target cells with viral or non-viral genetic material using an electroporation device,
wherein steps (a), (b), (c) and (d) are performed sequentially, in any order, or one or more of steps (a), (b), (c) and (d) are performed simultaneously with the remaining steps performed sequentially in any order.
2. The method of aspect 1, wherein the isolation technique of step (a) is acoustic separation, further wherein the purity of the target cells is increased in comparison to isolation of target cells with density, gradient and/or magnetic bead separation.
3. The method of aspect 1, wherein the isolation technique of step (a) comprises both acoustic separation and antibody conjugated labels, further wherein the purity of the target cells is increased in comparison to isolation of target cells with density, gradient and/or magnetic bead separation.
4. The method of aspect 2 or 3, wherein the increased purity of the target cells results at least in part from a reduced presence of monocytes among the target cells.
5. The method of any one of the preceding aspects, wherein after step (d) the target cells are expanded.
6. The method of any one of the preceding aspects, wherein after step (d) the target cells are cryopreserved.
7. The method of aspect 6, wherein the target cells are cryopreserved with a controlled rate freezer.
8. The method of any one of the preceding aspects, wherein the donor sourced material is selected from the group consisting of previously cryopreserved cells, leukapheresis product, peripheral whole blood, cord blood or any combination thereof.
9. The method of any one of the preceding aspects, wherein the target cells are transduced in a fluidic channel, wherein a fluid flow is provided to co-localizing binding of viral vector and target cells.
10. The method of any one of the preceding aspects, wherein the target cells comprise markers for CD3, CD4, CD8, CD14, CD19, CD25, CD27, CD28, CD34, CD56, CD69, CD95, CCR7, CD62L, CD45RA/RO, PD1, OX40, ICOS and any combination thereof.
11. The method of any one of the preceding aspects, wherein the target cells are isolated using antibody-conjugated magnetic beads wherein one or more antibodies have specificity for a marker selected from the group consisting of CD3, CD4, CD8, CD14, CD19, CD25, CD27, CD28, CD34, CD56, CD69, CD95, CCR7, CD62L, CD45RA/RO, PD1, OX40, ICOS and any combination thereof.
12. The method of any one of the preceding aspects, wherein the target cells are isolated using antibody-conjugated beads that respond to an acoustic field wherein one or more antibodies have specificity for a marker selected from the group consisting of CD3, CD4, CD8, CD14, CD19, CD25, CD27, CD28, CD34, CD56, CD69, CD95, CCR7, CD62L, CD45RA/RO, PD1, OX40, ICOS and any combination thereof.
13. The method of any one of the preceding aspects, wherein the target cells are isolated using antibody-conjugated beads that respond to a gravitational field and/or a centrifugation force wherein one or more antibodies have specificity for a marker selected from the group consisting of CD3, CD4, CD8, CD14, CD19, CD25, CD27, CD28, CD34, CD56, CD69, CD95, CCR7, CD62L, CD45RA/RO, PD1, OX40, ICOS and any combination thereof.
14. The method of any one of the preceding aspects, wherein the target cells are activated by contact with a soluble activating reagent selected from the group consisting of MACS® GMP T Cell TransAct™, CD137L, ImmunoCult™ Human CD3 Cell Activator, anti-CD28 antibody, anti-CD3 antibody, Interleukin-2, Interleukin-7, Interleukin-15, Interleukin-3, Interleukin-21, Thermogenesis X-Bacs and any combination thereof.
15. The method of any one of the preceding aspects, wherein the target cells are activated by contact with an insoluble activating reagent selected from the group consisting of Dynabeads™ Human T-Activator CD3/CD28, Cloudz Human T Cell Activation CD3/CD28 microspheres, CLOUDZ NK Cell Activation CD2/NKp46 microspheres, a microcarrier and any combination thereof.
16. The method of any one of the preceding aspects, wherein the target cells are activated in the absence of exogenous IL-2.
17. The method of any one of the preceding aspects, wherein the target cells are isolated with one or more antibody-conjugated magnetic beads.
18. The method of aspect 17, wherein the target cells are isolated with two or more antibody-conjugated magnetic beads, wherein each antibody-conjugated magnetic bead has specificity for a different target and the two or more antibody-conjugated magnetic beads are utilized sequentially for target cell selection.
19. The method of any one of the preceding aspects, wherein the target cells are isolated with one or more antibody-conjugated label.
20. The method of aspect 19, wherein the target cells are isolated with two or more antibody-conjugated labels, wherein each antibody-conjugated label has specificity for a different target and the two or more antibody-conjugated labels are utilized sequentially for target cell selection.
21. The method of aspect 19 or 20, wherein the label is a CLOUDZ label.
22. The method of any one of aspects 19 to 21, wherein the antibody has specificity for CD4.
23. The method of any one of aspects 19 to 21, wherein the antibody has specificity for CD8.
24. The method of any one of the preceding aspects, wherein steps (a) and (b) are performed simultaneously.
25. The method of any one of the preceding aspects, wherein steps (a) and (b) are performed simultaneously with anti-CD3 antibody, anti-CD28 antibody, CD137L, Interleukin-7, Interleukin-15, Interleukin-21, and any combination thereof.
26. The method of any one of the preceding aspects, wherein the isolation of the target cells in step (a) is performed under a condition selected from the group consisting of a static condition, a circulating condition, a mixing condition, a rocking condition, a suspension condition, a pressurized condition, a laminar flow condition, a turbulent flow condition and any combination thereof.
27. The method of any one of the preceding aspects, wherein the number of target cells is within the range of about 4e7 to about 1e10 cells.
28. The method of any one of the preceding aspects, wherein the number of target cells is within the range of 4e7 to 1e10 cells.
29. The method of any one of the preceding aspects, wherein the target cells are transduced in step (c) with viral vector within 0 to 72 hours of activation in step (b).
30. The method of any one of the preceding aspects, wherein the target cells are transduced in step (c) in the presence of an enhancing reagent selected from the group consisting of Retronectin, protamine sulfate, polybrene, LentiBOOST, ViralEntry™, Vectofusin-1 and any combination thereof.
31. The method of any one of the preceding aspects, wherein the target cells are transduced in step (c) in the absence of an exogenous enhancing reagent.
32. The method of any one of the preceding aspects, wherein the target cells are transduced in a fluidic channel, wherein the fluidic channel is comprised within a fluidic transmembrane device which provides an enclosed system with transmembrane flow and further provides for colocalization of the viral vector and the target cells onto a membrane with a molecular weight cut-off between about 200 kDa and about 1000 kDa.
33. The method of any one of the preceding aspects, wherein the transfection of step (d) precedes the activation of step (b) further wherein the target cells are contacted with plasmid DNA, mRNA, siRNA, or microRNA in step (d).
34. The method of any one of aspects 1-32, wherein the transfection of step (d) follows the contacting with an activating molecule of step (b) further wherein the target cells are contacted with plasmid DNA, mRNA, siRNA, or microRNA in step (d).
35. The method of any one of the preceding aspects, wherein the target cells are transfected with a cargo selected from the group consisting of a zinc finger nuclease mRNA, a TALEN mRNA, a CRISPR guided RNA/Cas ribonucleoprotein, or any combination thereof.
36. The method of any one of the preceding aspects, wherein the electroporation device is an enclosed system that generates pulsed waveforms to electroporate about 1e6 to about 1e10 target cells in batches using semi continuous flow.
37. The method of any one of the preceding aspects, wherein the electroporation device is an enclosed system that generates pulsed waveforms to electroporate about 1e6 to about 1e10 target cells in batches using continuous flow.
38. The method of any one of the preceding aspects, wherein the electric pulse profile for electroporation is a combination of 10 to 100 kV/m, 10 μs to 30 ms, for 1 to 30 pulses.
39. The method of any one of the preceding aspects, wherein the target cells are washed and concentrated into electroporation buffer or culture media using an enclosed centrifugation system to a cell concentration between about 2e7 and 1.5e8 cells per mL.
40. The method of any one of the preceding aspects, wherein the target cells are expanded after transfection using expansion feeding.
41. The method of aspect 40, wherein the expansion feeding is within an enclosed gas-permeable cell culture bag, a bioreactor, or any combination thereof.
42. The method of any one of aspects 1-39, wherein the target cells are expanded after transfection without using expansion feeding.
43. The method of any one of the preceding aspects, wherein the activating of step (b) is performed for up to 96 hours at about 37° C. and about 5% CO2.
44. The method of any one of the preceding aspects, further comprising performing a closed-system centrifugation wash to the target cells after the contacting with an activating molecule of step (b).
45. The method of any one of the preceding aspects, wherein the transducing of step (c) is performed with a CAR construct-encoded lentiviral vector, retroviral vector or adeno-associated vector using an enhancing reagent.
46. The method of aspect 45, wherein the enhancing reagent is selected from the group consisting of retronectin, protamine sulfate, polybrene, vectofusin-1, Sirion AdenoBOOST™, Sirion LentiBOOST and any combination thereof.
47. The method of any one of aspects 1-44, wherein the transducing of step (c) is performed with a CAR construct-encoded lentiviral vector, retroviral vector or adeno-associated vector without using an enhancing reagent.
48. The method of any one of aspects 45-47, wherein the transducing of step (c) is performed for 1 to 72 hours at a temperature between 15° C. to 37° C.
49. The method of aspect 1, wherein the activating of step (b) is performed sequentially prior to the transducing of step (c).
50. The method of any one of the preceding aspects, wherein the contacting with an activating molecule of step (b) is performed simultaneously with the transducing of step (c).
51. A method for manufacturing engineered human lymphocytes for cell therapies, comprising:
isolating target cells from apheresis of a healthy donor using an acoustic separation device; activating the target cells by stimulation of antibody receptors in the presence of IL-2 for up to 96 hours in an enclosed vessel;
washing activated target cells using a buffer exchange module;
transfecting the target cells with genetic or non-genetic material using an electroporation device;
transducing the target cells after transfection with construct-encoded lentiviral vectors or retroviral vectors with retronectin in the enclosed vessel;
expanding the target cells after transduction to a desired dose;
depleting the target cells after expansion using a negative selection stepwise isolation step to deplete the unedited TCRab+ cells; and
combining the target cells with cryoprotectant reagents and buffers to create a final formulation.
52. The method of aspect 51, wherein the target cells express markers selected from the group consisting of CD3, CD4, CD8, CD14, CD19, CD25, CD27, CD28, CD56, CD69, CD95, CCR7, CD62L, CD45RA/RO, PD1, OX40, ICOS and any combination thereof.
53. The method of aspect 51 or 52, further comprising washing activated target cells using a buffer exchange to concentrate the cells between 15-100 e6 cells/mL in media.
54. The method of any one of aspects 51 to 53, wherein the buffer exchange module processes at least 1e8 cells per lot.
55. The method of any one of aspects 51 to 54, further comprising washing the target cells after expansion and before depletion using acoustic separation to achieve a cell concentration of 50-300 e9 cells/mL in 200-500 mL.
56. The method of any one of aspects 51 to 55, further comprising suspending the target cells after depletion in a cryopreservation media.

Other aspects and advantages of the invention will become apparent from the following detailed description, taken in conjunction with the accompanying drawings, illustrating the principles of the invention by way of example only.

### BRIEF DESCRIPTION OF THE DRAWINGS

The patent or application file contains at least one drawing executed in color. Copies of this patent or patent application publication with color drawings will be provided by the U.S. Patent and Trademark Office upon request and payment of the necessary fee.

The teachings claimed and/or described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 depicts multiple exemplary embodiments for acoustic separation and activation process workflows.

FIG. 2 depicts process flow embodiments for both integrated viral gene transfer and for integrated non-viral gene transfer.

FIG. 3 depicts a flow chart of an alternative embodiment for a process flow involving transfection prior to transduction.

FIG. 4 depicts various embodiments of the allogeneic electroporation and transduction process flows.

FIG. 5 depicts a system for the process flow embodiments describe for a allogeneic process flows.

FIG. 6 depicts an embodiment of a process flow for a cell manufacturing method comprising a soluble activator and static viral transduction.

FIG. 7 depicts an embodiment of a process flow for a cell manufacturing method comprising activation using antibody coated surface with static viral transduction.

FIG. 8 depicts an embodiment of a process flow for a cell manufacturing method comprising activation using antibody coated surface with fluidic viral transduction.

FIG. 9 depicts an embodiment of a process flow for a cell manufacturing method comprising activation using antibody coated surface with static viral transduction, electroporation and gene editing.

FIG. 10 depicts an embodiment of a process flow for a cell manufacturing method comprising electroporation and non-viral gene delivery, optionally comprising activation using antibody coated surface and cell expansion.

FIG. 11 depicts an embodiment of a process flow for a cell manufacturing method comprising electroporation, and non-viral gene delivery, optionally comprising activation using antibody coated surface and cell expansion.

### DETAILED DESCRIPTION

The present disclosure addresses the need for an improved system and method for manufacturing engineered human lymphocytes where all or a combination of unit operations may be completed in an automated closed-system approach designed to create modular or continuous scale-down/scale-out unit operations. The below disclosure describes systems and methods that may consolidate raw materials and single-use kits, reduce labor and clean-room environmental monitoring costs associated with quality control, quality assurance, sterility, and process deviations. In certain embodiments, the system and method allow for consolidation of process equipment and unit operations enabled that allows for economies of scale and scale-up/scale-out to increase run rates, suite capacity, and reduce lot release time.

It will be understood that descriptions herein are exemplary and explanatory only and are not restrictive of the invention as claimed. In this application, the use of the singular includes the plural unless specifically stated otherwise.

All documents, or portions of documents, cited in this application, including but not limited to patents, patent applications, articles, books, and treatises, are hereby expressly incorporated by reference in their entirety for any purpose. As utilized in accordance with the present disclosure, the following terms, unless otherwise indicated, shall be understood to have the following meanings:

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” may mean within one or more than one standard deviation per the practice in the art. “About” or “approximately” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Systeme International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. Exemplary routes of administration for the compositions disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering may also be performed, for example, once, a plurality of times, and/or over one or more extended periods. In one embodiment, the CAR T cell treatment is administered via an “infusion product” comprising CAR T cells.

The term “antibody” (Ab) includes, without limitation, a glycoprotein immunoglobulin which binds specifically to an antigen. In general, an antibody may comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions may be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system.

Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above. In some embodiments, antibodies described herein refer to polyclonal antibody populations.

An “antigen binding molecule,” “antigen binding portion,” or “antibody fragment” refers to any molecule that comprises the antigen binding parts (e.g., CDRs) of the antibody from which the molecule is derived. An antigen binding molecule may include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In some embodiments, the antigen binding molecule binds to CD19. In further embodiments, the antigen binding molecule is an antibody fragment that specifically binds to the antigen, including one or more of the complementarity determining regions (CDRs) thereof. In further embodiments, the antigen binding molecule is a single chain variable fragment (scFv). In some embodiments, the antigen binding molecule comprises or consists of avimers.

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, may serve as an antigen. An antigen may be endogenously expressed, i.e. expressed by genomic DNA, or may be recombinantly expressed. An antigen may be specific to a certain tissue, such as a cancer cell, or it may be broadly expressed. In addition, fragments of larger molecules may act as antigens. In some embodiments, antigens are tumor antigens.

The term “neutralizing” refers to an antigen binding molecule, scFv, antibody, or a fragment thereof, that binds to a ligand and prevents or reduces the biological effect of that ligand. In some embodiments, the antigen binding molecule, scFv, antibody, or a fragment thereof, directly blocks a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

The term “allogeneic” refers to any material derived from one individual which is then introduced to another individual of the same species, e.g., allogeneic T cell transplantation.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. In this application, the term cancer is synonymous with malignancy. Examples of cancers that may be treated by the methods disclosed herein include, but are not limited to, cancers of the immune system including lymphoma, leukemia, myeloma, and other leukocyte malignancies. In some embodiments, the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, [add other solid tumors] multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers including those induced by asbestos, other B cell malignancies, and combinations of said cancers. In some embodiments, the cancer is multiple myeloma. In some embodiments, the cancer is NHL. The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

An “anti-tumor effect” as used herein, refers to a biological effect that may present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect may also refer to the prevention of the occurrence of a tumor, e.g., a vaccine.

A “cytokine,” as used herein, refers to a non-antibody protein that is released by one cell in response to contact with a specific antigen, wherein the cytokine interacts with a second cell to mediate a response in the second cell. “Cytokine” as used herein is meant to refer to proteins released by one cell population that act on another cell as intercellular mediators. A cytokine may be endogenously expressed by a cell or administered to a subject. Cytokines may be released by immune cells, including macrophages, B cells, T cells, and mast cells to propagate an immune response. Cytokines may induce various responses in the recipient cell. Cytokines may include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

As used herein, “chimeric receptor” refers to an engineered surface expressed molecule capable of recognizing a particular molecule. Chimeric antigen receptors (CARs) and engineered T cell receptors (TCRs), which comprise binding domains capable of interacting with a particular tumor antigen, allow T cells to target and kill cancer cells that express the particular tumor antigen. In one embodiment, the T cell treatment is based on T cells engineered to express a chimeric antigen receptor (CAR) or a T cell receptor (TCR), which comprises (i) an antigen binding molecule, (ii) a costimulatory domain, and (iii) an activating domain. The costimulatory domain may comprise an extracellular domain, a transmembrane domain, and an intracellular domain, wherein the extracellular domain comprises a hinge domain, which may be truncated.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. Such terms can be used interchangeably. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T cells play a major role in cell-mediated-immunity (no antibody involvement). Its T cell receptors (TCR) differentiate themselves from other lymphocyte types. The thymus, a specialized organ of the immune system, is primarily responsible for the T cell's maturation. There are six types of T cells, namely: Helper T cells (e.g., CD4+ cells), Cytotoxic T cells (also known as TC, cytotoxic T lymphocyte, CTL, T-killer cell, cytolytic T cell, CD8+ T cells or killer T cell), Memory T cells ((i) stem memory TSCM cells, like naive cells, are CD45RO−, CCR7+, CD45RA+, CD62L+(L-selectin), CD27+, CD28+ and IL-7Ra+, but they also express large amounts of CD95, IL-2Rβ, CXCR3, and LFA-1, and show numerous functional attributes distinctive of memory cells); (ii) central memory TCM cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T cells (Tregs, suppressor T cells, or CD4+CD25+ regulatory T cells), Natural Killer T cells (NKT) and Gamma Delta T cells. B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy may include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. Nos. 7,741,465, 6,319,494, 5,728,388, and International Publication No. WO 2008/081035. In some embodiments, the immunotherapy comprises CAR T cell treatment. In some embodiments, the CAR T cell treatment product is administered via infusion.

The T cells of the immunotherapy may come from any source known in the art. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety.

The term “engineered Autologous Cell Therapy,” or “eACT™,” also known as adoptive cell transfer, is a process by which a patient's own T cells are collected and subsequently genetically altered to recognize and target one or more antigens expressed on the cell surface of one or more specific tumor cells or malignancies. T cells may be engineered to express, for example, chimeric antigen receptors (CAR). CAR positive (+) T cells are engineered to express an extracellular single chain variable fragment (scFv) with specificity for a particular tumor antigen linked to an intracellular signaling part comprising at least one costimulatory domain and at least one activating domain. The CAR scFv may be designed to target, for example, CD19, which is a transmembrane protein expressed by cells in the B cell lineage, including all normal B cells and B cell malignances, including but not limited to diffuse large B-cell lymphoma (DLBCL) not otherwise specified, primary mediastinal large B-cell lymphoma, high grade B-cell lymphoma, and DLBCL arising from follicular lymphoma, NHL, CLL, and non-T cell ALL. Example CAR T cell therapies and constructs are described in U.S. Patent Publication Nos. 2013/0287748, 2014/0227237, 2014/0099309, and 2014/0050708, and these references are incorporated by reference in their entirety.

A “patient” as used herein includes any human who is afflicted with a cancer (e.g., a lymphoma or a leukemia). The terms “subject” and “patient” are used interchangeably herein.

As used herein, the term “in vitro cell” refers to any cell which is cultured ex vivo. In particular, an in vitro cell may include a T cell. The term “in vivo” means within the patient.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that may comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex that specifically binds with a cognate stimulatory ligand present on an antigen present cell. A “stimulatory ligand” is a ligand that when present on an antigen presenting cell (e.g., an APC, a dendritic cell, a B-cell, and the like) may specifically bind with a stimulatory molecule on a T cell, thereby mediating a primary response by the T cell, including, but not limited to, activation, initiation of an immune response, proliferation, and the like. Stimulatory ligands include, but are not limited to, an anti-CD3 antibody, an MHC Class I molecule loaded with a peptide, a superagonist anti-CD2 antibody, and a superagonist anti-CD28 antibody.

A “costimulatory signal,” as used herein, refers to a signal, which in combination with a primary signal, such as TCR/CD3 ligation, leads to a T cell response, such as, but not limited to, proliferation and/or upregulation or down regulation of key molecules.

A “costimulatory ligand,” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand may include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. In certain embodiments, a co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD33, CD45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD11a, CD11b, CD11c, CD11d, CDS, CEACAM1, CRT AM, DAP-10, DNAM1 (CD226), Fc gamma receptor, GADS, GITR, HVEM (LIGHTR), IA4, ICAM-1, ICOS, Ig alpha (CD79a), IL2R beta, IL2R gamma, IL7R alpha, integrin, ITGA4, ITGA6, ITGAD, ITGAE, ITGAL, ITGAM, ITGAX, ITGB2, ITGB7, ITGB1, KIRDS2, LAT, LFA-1, LIGHT (tumor necrosis factor superfamily member 14; TNFSF14), LTBR, Ly9 (CD229), lymphocyte function-associated antigen-1 (LFA-1 (CD11a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions. Similarly, the term “increasing” indicates any change that is higher than the original value. “Increasing,” “higher,” and “lower” are relative terms, requiring a comparison between pre- and post-measurements and/or between reference standards. In some embodiments, the reference values are obtained from those of a general population, which could be a general population of patients. In some embodiments, the reference values come quartile analysis of a general patient population.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In another embodiment, “treatment” or “treating” includes a complete remission.

As used herein, the term “polyfunctional T cells” refers to cells co-secreting at least two proteins from a pre-specified panel per cell coupled with the amount of each protein produced (i.e., combination of number of proteins secreted and at what intensity). In some embodiments, a single cell functional profile is determined for each evaluable population of engineered T cells. Profiles may be categorized into effector (Granzyme B, IFN-γ, MIP-1α, Perforin, TNF-α, TNF-β), stimulatory (GM-CSF, IL-2, IL-5, IL-7, IL-8, IL-9, IL-12, IL-15, IL-21), regulatory (IL-4, IL-10, IL-13, IL-22, TGF-β1, sCD137, sCD40L), chemoattractive (CCL-11, IP-10, MIP-1β, RANTES), and inflammatory (IL-1b, IL-6, IL-17A, IL-17F, MCP-1, MCP-4) groups. In some embodiments, the functional profile of each cell enables the calculation of other metrics, including a breakdown of each sample according to cell polyfunctionality (i.e., what percentage of cells are secreting multiple cytokines versus non-secreting or monofunctional cells), and a breakdown of the sample by functional groups (i.e., which mono- and polyfunctional groups are being secreted by cells in the sample, and their frequency).

As used herein, the term “quartile” or “quadrant” is a statistical term describing a division of observations into four defined intervals based upon the values of the data and how they compare to the entire set of observations.

As used herein, the term “Study day 0” is defined as the day the subject received the first CAR T cell infusion. The day prior to study day 0 will be study day −1. Any days after enrollment and prior to study day −1 will be sequential and negative integer-valued.

As used herein, the term “objective response” refers to complete response (CR), partial response (PR), or non-response. Criteria are based on the revised IWG Response Criteria for Malignant Lymphoma.

As used herein, the term “complete response” refers to complete resolution of disease, which becomes not detectable by radio-imaging and clinical laboratory evaluation. No evidence of cancer at a given time.

As used herein, the term “partial response” refers to a reduction of greater than 30% of tumor without complete resolution. Criteria are based on the revised IWG Response Criteria for Malignant Lymphoma where PR is defined as “At least a 50% decrease in sum of the product of the diameters (SPD) of up to six of the largest dominant nodes or nodal masses. These nodes or masses should be selected according to all of the following: they should be clearly measurable in at least 2 perpendicular dimensions; if possible they should be from disparate regions of the body; and they should include mediastinal and retroperitoneal areas of disease whenever these sites are involved.

As used herein, the term “non-response” refers to the subjects who had never experienced CR or PR post CAR T cell infusion.

As used herein, the term “durable response” refers to the subjects who were in ongoing response at least by one year follow up post CAR T cell infusion 6 months f/u is utilized only for Z1, C3 as there is no longer f/u available for this cohort. Nevertheless, the conclusions remain same.

As used herein, the term “relapse” refers to the subjects who achieved a complete response (CR) or partial response (PR) and subsequently experienced disease progression.

As used herein, the expansion and persistence of CAR T cells in peripheral blood may be monitored by qPCR analysis, for example using CAR-specific primers for the scFv portion of the CAR (e.g., heavy chain of a CD19 binding domain) and its hinge/CD28 transmembrane domain. Alternatively, it may be measured by enumerating CAR cells/unit of blood volume.

As used herein, the scheduled blood draw for CAR T cells may be before CAR T cell infusion, Day 7, Week 2 (Day 14), Week 4 (Day 28), Month 3 (Day 90), Month 6 (Day 180), Month 12 (Day 360), and Month 24 (Day 720).

As used herein, the “peak of CAR T cell” is defined as the maximum absolute number of CAR+PBMC/μL in serum attained after Day 0.

As used herein, the “time to Peak of CAR T cell” is defined as the number of days from Day 0 to the day when the peak of CAR T cell is attained.

As used herein, the “Area Under Curve (AUC) of level of CAR T cell from Day 0 to Day 28” is defined as the area under the curve in a plot of levels of CAR T cells against scheduled visits from Day 0 to Day 28. This AUC measures the total levels of CAR T cells overtime.

As used herein, the scheduled blood draw for cytokines is before or on the day of conditioning chemotherapy (Day −5), Day 0, Day 1, Day 3, Day 5, Day 7, every other day if any through hospitalization, Week 2 (Day 14), and Week 4 (Day 28).

As used herein, the “baseline” of cytokines is defined as the last value measured prior to conditioning chemotherapy.

As used herein, the fold change from baseline at Day X is defined as

$$\frac{Cytokine level at Day X - Baseline}{Baseline}$$

As used herein, the “peak of cytokine post baseline” is defined as the maximum level of cytokine in serum attained after baseline (Day −5) up to Day 28.

As used herein, the “time to peak of cytokine” post CAR T cell infusion is defined as the number of days from Day 0 to the day when the peak of cytokine was attained.

As used herein, the “Area Under Curve (AUC) of cytokine levels” from Day −5 to Day 28 is defined as the area under the curve in a plot of levels of cytokine against scheduled visits from Day −5 to Day 28. This AUC measures the total levels of cytokine overtime. Given the cytokine and CAR+ T cell are measured at certain discrete time points, the trapezoidal rule may be used to estimate the AUCs.

It will be appreciated that chimeric antigen receptors (CARs or CAR-Ts) are, and T cell receptors (TCRs) may, be genetically engineered receptors. These engineered receptors may be readily inserted into and expressed by immune cells, including T cells in accordance with techniques known in the art. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell.

CARs may be engineered to bind to an antigen (such as a cell-surface antigen) by incorporating an antigen binding molecule that interacts with that targeted antigen. An “antigen binding molecule” as used herein means any protein that binds a specified target molecule. Antigen binding molecules include, but are not limited to antibodies and binding parts thereof, such as immunologically functional fragments. Peptibodies (i.e., Fc fusion molecules comprising peptide binding domains) are another example of suitable antigen binding molecules.

Preferably, target molecules may include, but are not limited to, blood borne cancer-associated antigens. Non-limiting examples of blood borne cancer-associated antigens include antigens associated with one or more cancers selected from the group consisting of acute myeloid leukemia (AML), chronic myelogenous leukemia (CML), chronic myelomonocytic leukemia (CMML), juvenile myelomonocytic leukemia, atypical chronic myeloid leukemia, acute promyelocytic leukemia (APL), acute monoblastic leukemia, acute erythroid leukemia, acute megakaryoblastic leukemia, lymphoblastic leukemia, B-lineage acute lymphoblastic leukemia, B-cell chronic lymphocytic leukemia, B-cell non-Hodgkin's lymphoma, myelodysplastic syndrome (MDS), myeloproliferative disorder, myeloid neoplasm, myeloid sarcoma), and Blastic Plasmacytoid Dendritic Cell Neoplasm (BPDCN).

In some embodiments, the antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CSPG4, CTLA-4, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, mesothelin, MN-CA IX, MUC-1, mut hsp72, mutated p53, mutated p53, mutated ras, neutrophil elastase, NKG2D, Nkp30, NY-ESO-1, p53, PAP, prostase, prostase specific antigen (PSA), prostate carcinoma tumor antigen-1 (PCTA-1), prostate-specific antigen, prostein, PSMA, RAGE-1, ROR1, RU1, RU2 (AS), surface adhesion molecule, surviving and telomerase, TAG-72, the extra domain A (EDA) and extra domain B (EDB) of fibronectin and the Al domain of tenascin-C(TnC Al), thyroglobulin, tumor stromal antigens, vascular endothelial growth factor receptor-2 (VEGFR2), virus-specific surface antigen such as an HIV-specific antigen (such as HIV gpl20), as well as any derivate or variant of these surface markers.

In some embodiments, target molecules may include viral infection-associated antigens. Viral infections of the present disclosure may be caused by any virus, including, for example, HIV. This list of possible target molecules is not intended to be exclusive.

The TCRs of the present disclosure may bind to, for example, a tumor-associated antigen. As used herein, “tumor-associated antigen” refers to any antigen that is associated with one or more cancers selected from the group consisting of: adrenocortical carcinoma, anal cancer, bladder cancer, bone cancer, brain cancer, breast cancer, carcinoid cancer, carcinoma, cervical cancer, colon cancer, endometrial cancer, esophageal cancer, extrahepatic bile duct cancer, extracranial germ cell cancer, eye cancer, gallbladder cancer, gastric cancer, germ cell tumor, gestational trophoblastic tumor, head and neck cancer, hypopharyngeal cancer, islet cell carcinoma, kidney cancer, large intestine cancer, laryngeal cancer, leukemia, lip and oral cavity cancer, liver cancer, lung cancer, lymphoma, malignant mesothelioma, Merkel cell carcinoma, mycosis fungoides, myelodysplastic syndrome, myeloproliferative disorders, nasopharyngeal cancer, neuroblastoma, oral cancer, oropharyngeal cancer, osteosarcoma, ovarian epithelial cancer, ovarian germ cell cancer, pancreatic cancer, paranasal sinus and nasal cavity cancer, parathyroid cancer, penile cancer, pituitary cancer, plasma cell neoplasm, prostate cancer, rhabdomyosarcoma, rectal cancer, renal cell cancer, transitional cell cancer of the renal pelvis and ureter, salivary gland cancer, Sezary syndrome, skin cancers, small intestine cancer, soft tissue sarcoma, stomach cancer, testicular cancer, thymoma, thyroid cancer, urethral cancer, uterine cancer, vaginal cancer, vulvar cancer, and Wilms' tumor.

In certain embodiments, the present disclosure may be suitable for target molecule to hematologic cancer. In some embodiments, the cancer is of the white blood cells. In other embodiments, the cancer is of the plasma cells. In some embodiments, the cancer is leukemia, lymphoma, or myeloma. In certain embodiments, the cancer is acute lymphoblastic leukemia (ALL) (including non T cell ALL), acute lymphoid leukemia (ALL), and hemophagocytic lymphohistocytosis (HLH)), B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia (CML), chronic or acute granulomatous disease, chronic or acute leukemia, diffuse large B cell lymphoma, diffuse large B cell lymphoma (DLBCL), follicular lymphoma, follicular lymphoma (FL), hairy cell leukemia, hemophagocytic syndrome (Macrophage Activating Syndrome (MAS), Hodgkin's Disease, large cell granuloma, leukocyte adhesion deficiency, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome (MDS), myeloid diseases including but not limited to acute myeloid leukemia (AML), non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), primary mediastinal large B cell lymphoma (PMBCL), small cell- or a large cell-follicular lymphoma, splenic marginal zone lymphoma (SMZL), systemic amyloid light chain amyloidosis, T-cell acute lymphoid leukemia (TALL), T-cell lymphoma, transformed follicular lymphoma, Waldenstrom macroglobulinemia, or a combination thereof.

The TCRs of the present disclosure may also bind to a viral infection-associated antigen. Viral infection-associated antigens include antigens associated with any viral infection, including, for example, viral infection caused by HIV.

Various embodiments are described in further detail in the following subsections.

### Autologous Car-T/TCR System and Method

Provided herein are systems and methods for manufacturing engineered human lymphocytes in a closed-system modular or continuous fashion for autologous cell therapy targeting hematological or solid tumor indications via CAR-T, TCR, NK, iPSC or similar cell composition methodologies of clinical therapeutic value. Targeted cells of interest are isolated via positive or negative selection using density gradient, magnetic bead, or acoustic forces to obtain a mass of pure cells ready for activation via environmental pressures or antibody co-stimulation, the later which is envisioned to be done sequentially or concurrently with isolation using antibody conjugated or physical coated beads, labels, surfaces, or particles bound to target cells. Activated cells are washed via centrifugation or buffer exchange and transfected or transduced through physical co-localization of cell and vector (RVV, LVV) or DNA/RNA capsule. Transfection and/or transduction is designed to be done with or without pre reagent coating of culture surfaces and either sequentially or concurrently with activation. Then, transduced cells may be pre-washed into an expansion step utilizing various batch, batch-fed, perfusion, and solera methods to obtain a sufficient mass of cells to meet dose. Final formulation is achieved through addition of cryoprotectant reagents and buffers to the cell mass at a specified ratio via buffer exchange, acoustic separation, centrifugation, gravitation, pumping, or syringe fluid handling techniques.

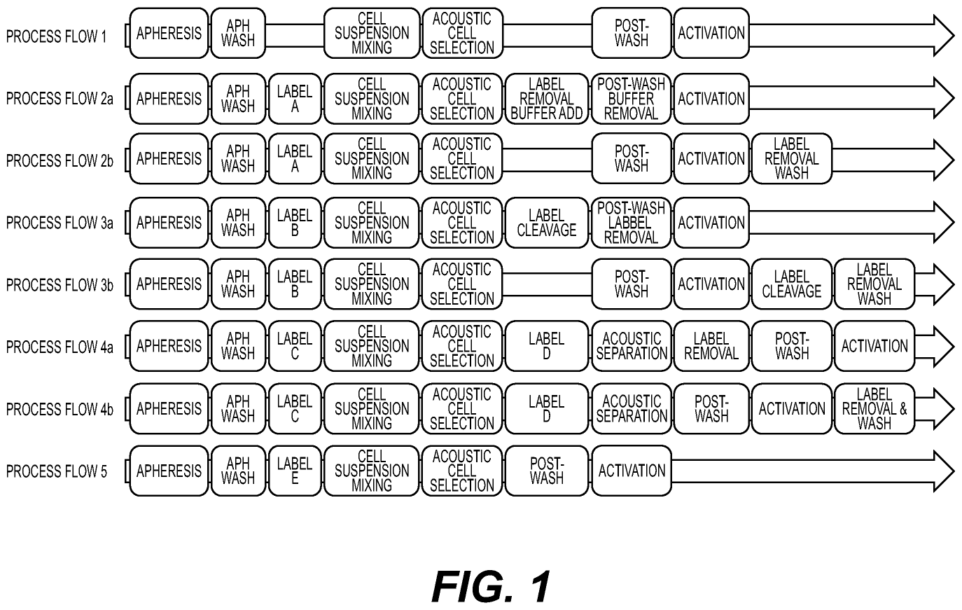

With reference to FIG. 1, there are shown multiple exemplary embodiments for an acoustic separation and activation process workflows. Various acoustic cell selection and activation steps shown in FIG. 1 are applicable to both autologous and allogeneic process flows that will be described below.

Exemplary process flow 1 shown in FIG. 1 is the baseline acoustic cell selection process, that includes apheresis collection, apheresis wash, cell suspension and mixing, acoustic cell selection, post wash and cell activation. Other exemplary process flows 2a through 5 shown in FIG. 1 differ from the process flow 1. For example, process flow 2a includes a label step pre-selection and a label removal step pre-activation. Also, in process flow 2b, the label removal step is moved to post-activation to enable cell selection and activation to occur at the same time. In one embodiment, process flows 2a and 2b use a label “A” such as a hydrogel that can easily be dissolved with a buffer such as EDTA. Continuing, exemplary process flow 3a is similar to process flow 2a except using a label “B” such as a polymer that is irreversibly conjugated with an Ab that requires a cleavage step post cell selection to free the target cell from the Ab. A post-wash step is included to remove the unbound label and exchange the selection buffer with culture media in process flow 3a. Exemplary process flow 3b is similar to process flow 3a except that the label removal (cleavage and wash) occurs after activation to enable cell selection and activation to occur at the same time. Exemplary process flow 4a depicts sequential cell selection using primary, secondary, etc. labelling, and exemplary process flow 4b depicts sequential cell selection with label removal post activation. Exemplary process flow 5 is similar to process flows 2a through 4 except there is no label removal step and process flow 5 relies on hydrolysis over time to dissolve the label.

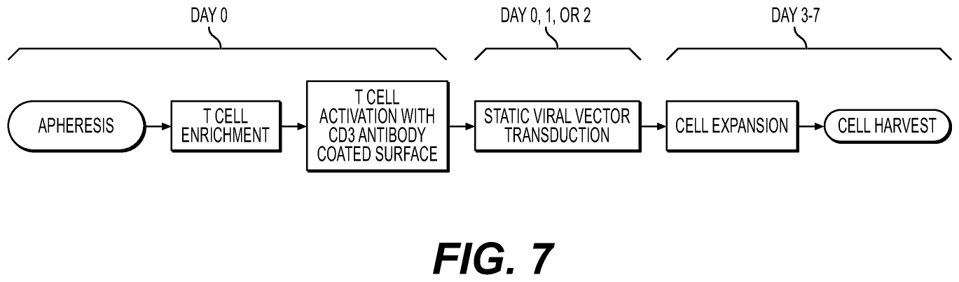

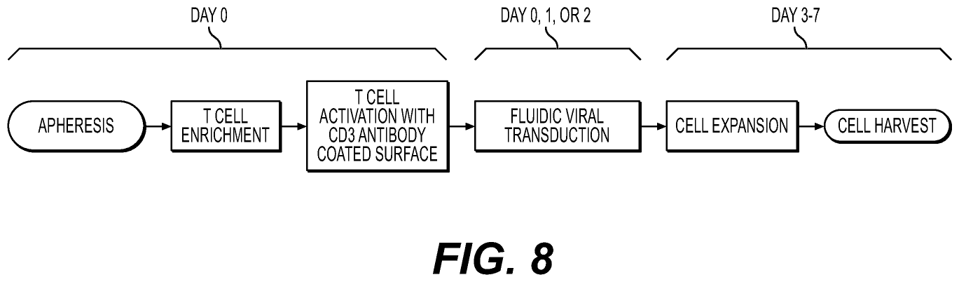

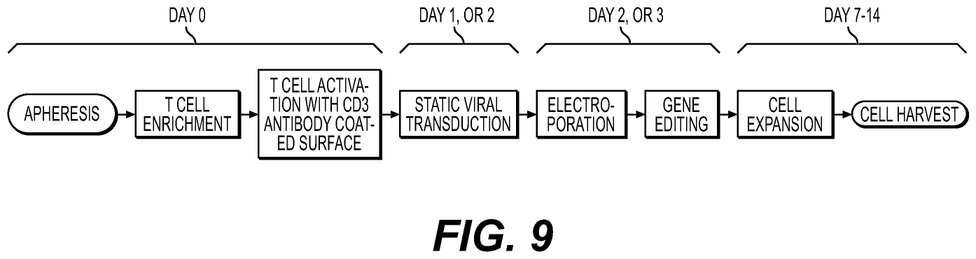

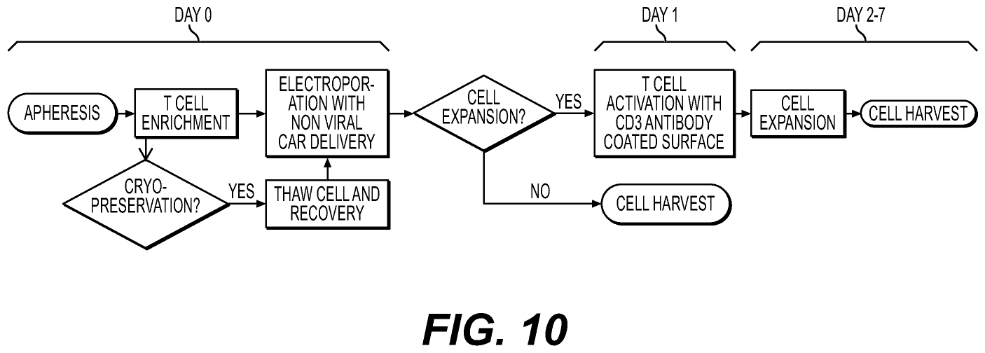

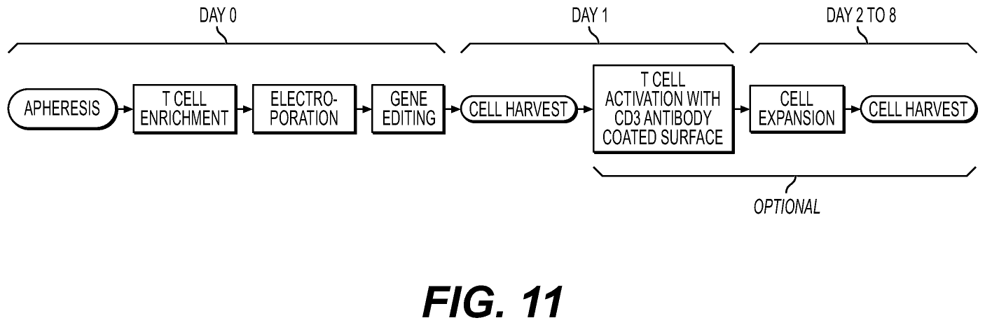

In FIG. 7 is shown an exemplary, improved process flow comprising activation using antibody coated surface with static viral transduction. In FIG. 8 is shown an exemplary, improved process flow comprising activation using antibody coated surface with fluidic viral transduction. In FIG. 9 is shown an exemplary, improved process flow comprising activation using antibody coated surface with static viral transduction, electroporation and gene editing. In FIG. 10 is shown an exemplary, improved process flow comprising electroporation, and non-viral gene delivery, optional activation using antibody coated surface and cell expansion. In FIG. 11 is shown an exemplary, improved process flow comprising electroporation, and non-viral gene delivery, optional activation using antibody coated surface and cell expansion. It will be understood that the process flows provided by non-limiting embodiment in the examples are modular in nature and can be varied in order and duration in order to achieve phenotypic benefits in lymphocyte manufacture, including but not limited to improved viability, efficacy, CAR expression, expansion capacity and various characteristics of less differentiated cells.

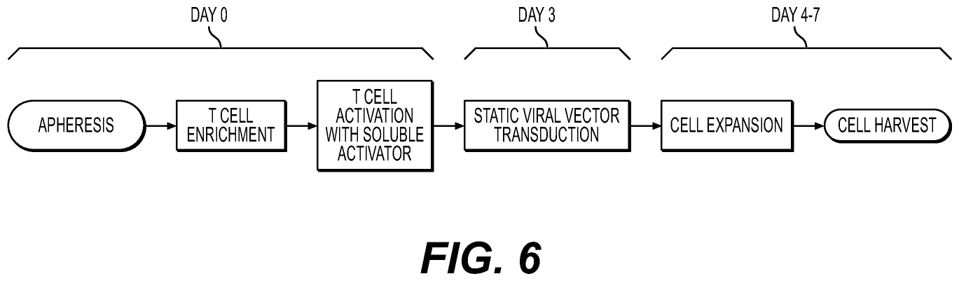

The following exemplary description will focus on process flow 1 but will be applicable to process flows 2a through 5 as well as the process flows of FIGS. 6-11.

### Cell Selection

In one embodiment of the system and method for manufacturing engineered human lymphocytes for cell therapies, apheresis is conducted on a patient at a clinic in order to collect cells in an autologous process. An apheresis starting material of typically 150 mL-300 mL containing typically between 3-35 e9 nucleated cells undergoes a preliminary wash (centrifugation, buffer exchange) to standardize the volume and reduce remnant RBCs, platelets, and cell debris that may interfere with the isolation selection step. In certain embodiments, if a cell sub-population of clinical relevance is desired, a pre-labelling may be introduced using antibody conjugated beads, labels, or particles. The labels bind to the cells of interest (positive selection) or to the cells to be depleted (negative selection) and are designed to have a particular physical characteristic that enhances or diminishes the cells response to the selection force (gravity, magnetic, acoustic, density, etc.). In addition to sub-population targeting, label binding is designed to improve recovery, purity, isolation sensitivity, cell selection robustness (i.e., apheresis shelf-life), viability, and throughput. In certain embodiments, the label binding step includes performing cell binding kinetics under 1 hour, label dissolution under 7 days. Furthermore, the label binding step may include downstream label removal or cleavage in less than 2 steps and in phycological conditions under 1 hour, using conventional methods, such as centrifugation, buffer exchange, acoustics. In certain embodiments, any unbound labels may be removed by closed-system wash steps. Also, the label binding step may be compatible with cell selection buffer or media.

In one embodiment, to enhance cell-label binding, the cells and labels are combined at a cell to label ratio for a defined time under static, rocking, shaking, circulating or other homogenous mixing techniques. Cell to label ratio can be defined by w/w, w/v, or v/v. In one embodiment, the cell to label ratio may range from 1-35 e9 TVC per 4-12 mL label. Cell types targeted for labelling may include markers for CD3, CD4, CD8, CD14, CD19, CD25, CD27, CD28, CD56, CD69, CD95, CCR7, CD62L, CD45RA/RO, PD1, OX40, ICOS expression or a combination. Sequential labelling for primary, secondary, tertiary, etc. targets based on cell composition percentage (CD3 followed by CD14) to avoid non-selective binding to non-target cells that express target antigen.

In certain embodiments, labelled cells may be maintained in a homogenous suspension as the cells enter the cell isolation step, using a device that performs cell enrichment using ultrasonic separation techniques, with voltage, frequency, flow rate and relevant parameters set to achieve a processing throughput of not less than 5 e9 nucleated cells per hour. Similar examples of acoustic cell separators are the Applikon Biosep cell retention device used for perfusion cultures and the Pall FloDesign Sonic Ekko cell processing system. Cells are introduced into a fluidic channel under ultrasonic excitation where the cells separate from the media based on the waveform pattern that propagates through the fluidic channel. Cells of different subtypes may also be separated based on their differing density, compressibility, and size characteristics. The fluidic channel may have multiple waves or a single wave that transverses the channel. Co-flow, density modification buffers, and cell labelling can be added to achieve the desired cell isolation performance criteria. Applications of sonic cell separation are acoustophoresis, RBC and platelet removal, cell depletion, cell concentration, and culture washes, and may be used to replace existing methods such as centrifugation, Ficoll separation, magnetic bead isolation, or filtration.

One embodiment includes a post cell selection label removal step, which involves buffer or reagent additives to enzymatically or chemically cleave the cells from the labels, followed by another acoustic separation, buffer exchange, or wash step to remove the labels. The label removal step may occur in an acoustic separation output bag or in an activation bag if the label removal step occurs post activation. In this way the cells of interest do not need to be transferred into a separate closed vessel. Another embodiment skips the label removal step and allows dissolution through hydrolysis over the duration of the process. This latter technique simplifies unit operations and enables direct to activation methodology without having to re-add co-stimulatory antibodies, i.e. selection and activation at the same time, (1-time reagent addition).

In one embodiment, a final post cell selection wash step may be included to exchange the cell isolation media with culture growth media for activation or exchanged with buffers for cryopreservation.

### Activation

One embodiment of the system and method described herein includes T lymphocyte activation or stimulation. Cells are activated with antibody conjugated beads or particles or activated in situ with antibody-coated surface in an enclosed vessel. This step may occur in a culture bag or other closed vessel after the cells are transferred from the acoustic separation device or output bag in a closed system. At-scale activation culture volumes range from 100 mL-6 L using a total of 0.1 to 9.0 e9 selected or enriched target cells for a time scale of hours or days (i.e., up to 96 hours) at 37° C. and 5% CO2. Environmental pressures may also be applied to co-stimulate activation in the absence of or reduced use of antibody enhancers.

One embodiment of the system and method includes a post-activation closed-system centrifugation wash or buffer exchange step. If desired, a concentration step is performed prior to the buffer exchange to achieve throughput targets. Concentration for example can be done using the Sepax, Sefia, K-Sep, or LOVO would target a final volume below 300 mL at a cell density up to 150 e6 selected cells/mL. Concentrated cells may go into a pre-mixer to maintain a homogenous suspension going into the buffer exchange. The buffer exchange parameters are selected to achieve a throughput greater than 2.5 e9 selected cells per hour.

### Transduction

One embodiment of the system and method includes a viral transduction step where pre or post-activated washed cells are transduced with construct-encoded lentiviral vectors (LVV), retroviral vectors (RVV), or adeno-associated vectors (AAV) using enhancing reagents alone or in combination (e.g. retronectin, protamine sulfate, polybrene, vectofusin-1, Sirion AdenoBOOST™, Sirion LentiBOOST), or enhancer-free physical co-localization (e.g. centrifugation-based spinoculation by Cytiva Sepax C-pro or Miltenyi Prodigy). In another embodiment, an enhancer-free physical co-localization transduction step based on a membrane pin-and-release mechanism where the cells and vector are co-localized onto a membrane under flow pressure using tangential flow hollow fiber filters. One embodiment may combine physical co-localization with single or multiple enhancer reagents. The viral vector-based gene delivery occurs at a cell to vector ratio designed to A) achieve a desired transduction efficiency (e.g., genomic integration rate) of at least 1.5 times greater than using enhancing reagents alone, and B) achieve a greater than 1.5-fold improvement with a greater than 50% reduction in vector particle units compared to using enhancing reagents alone.

The target cells for this embodiment may include peripheral blood mononucleated cells (PBMCs), T cells, NK cells, NK T cells, monocyte/macrophages, hematopoietic stem cells (HSC), HSC-derived cells, iPSC or iPSC-derived target cells. The target cells can be stimulated, or unstimulated, prior to transduction. Prior to the transduction, the target cells can be buffer exchanged or concentrated in culture media (e.g. Thermo Fisher CTS OpTimizer medium, Miltenyi TexMACS™ Medium, Lonza X-VIVO™ 15 Medium, and FUJIFILM PRIME-XV T Cell medium), or balanced buffers (e.g. PBS, DPBS, HBSS), using Cytiva Sepax, Cytiva Sefia, or Miltenyi CliniMACS Plus or Prodigy, and Fresenius Kabi Lovo systems. The viral transduction may take place in situ in the cell culture vessel (e.g. gas-permeable cell culture bags, cell culture plates, flasks, G-Rex cell culture platform), or microfluidic devices using membrane pin-and-release tangential flow hollow fiber filters, or bioreactor systems (e.g. Cytiva Xuri WAVE system, Cytiva Xcellrex single-use system, Thermo Fisher single-use system). The viral transduction can take place from 1 to 72 hours following the target cell stimulation or activation. The volume of viral vector is controlled at a target multiplicity of infection (transducing viral particle units per cell) or scaling factors involving volume height, surface area and incorporated into the transduction system or the culture system. Transduction may take from 1 hour to 72 hours at temperature ranges from 15° C. to 37° C.

### Transfection

In one embodiment, the system and method include the step of electroporation of cells to transfer genetic or non-genetic material i.e. cargo (e.g. DNA or RNA encoding transposon, endonuclease and CRISPR/Cas system, circular or linearized DNA, ribonucleoproteins etc.). The activated cells, or non-activated cells including T-cells, NK, NKT, B-cells, iPSC, macrophages, HSC, etc. cells), are centrifuged or buffer exchanged and suspended in electroporation buffer. The transgene-encoding plasmid DNA or mRNA, or DNA/RNA encoding host gene targeted cleaving endonuclease, or ribonucleoproteins are added into the system. The cells are electroporated using a cell electroporation system (e.g. Neon, Maxcyte, Nucleofector, DRAPER and others, etc.) The cell and genetic or non-genetic material mixture is subjected to defined electrical pulses resulting in transient opening of the cell membrane allowing passive or active diffusion of genetic or non-genetic material into the cells. The transfected cells are then transferred to growth media and allowed to recover for a defined time period. Recovered or non-recovered cells are then transferred to appropriate cell culture vessels including culture bags, G-rex bioreactors, Xuri Wave systems, or suspension culture bioreactors to allow for expansion in cell numbers. Multiple pre, during and post electroporation conditions and parameters including choice of buffers and media, viable cell density, cell viability, total number of viable cells, flow rates, concentration of genetic or non-genetic material, ratio of cells to cargo, purity of genetic material, contact time of cells and cargo to the electroporation buffer, design & choice of electrode material voltage, pulse number, pulse duration, pulse profile, measurement of transfection efficiency and parameters/conditions to optimize recovery and expansion post electroporation.

### Expansion

The transduced or transfected cells are transferred to a wash step (centrifugation, buffer exchange, acoustic), or directly to an expansion step at specified seed density ranges designed to achieve required growth rates. Expansion feeding methods may include batch, batch-fed, perfusion, solera or other scale up/out methods in culture bags, flasks, plates, vessels, wave or stirred tank suspension bioreactors.

### Final Formulation

A final formulation step occurs after a sufficient mass of target engineered cells are achieved to meet dose. Formulation involves a harvest wash step using the following options: centrifugation resuspension via Cytiva Sepax CultureWash or Sefia FlexCell, perfusion dilution via Cytiva Xuri WAVE system or Applikon Biosep cell retention, or buffer exchange inertial flow fluid dynamics. After the wash step, a dose specific post-wash volume is combined with cryoprotectant reagents and buffers at a specified ratio in a closed vessel. For final formulation, the step may occur in using the Terumo FINIA system, the buffer exchange inertial flow device, or traditional manual methods using gravitational, pump, or syringe fluid handling techniques. The final product bags are cryopreserved for storage, shipment, and later use.

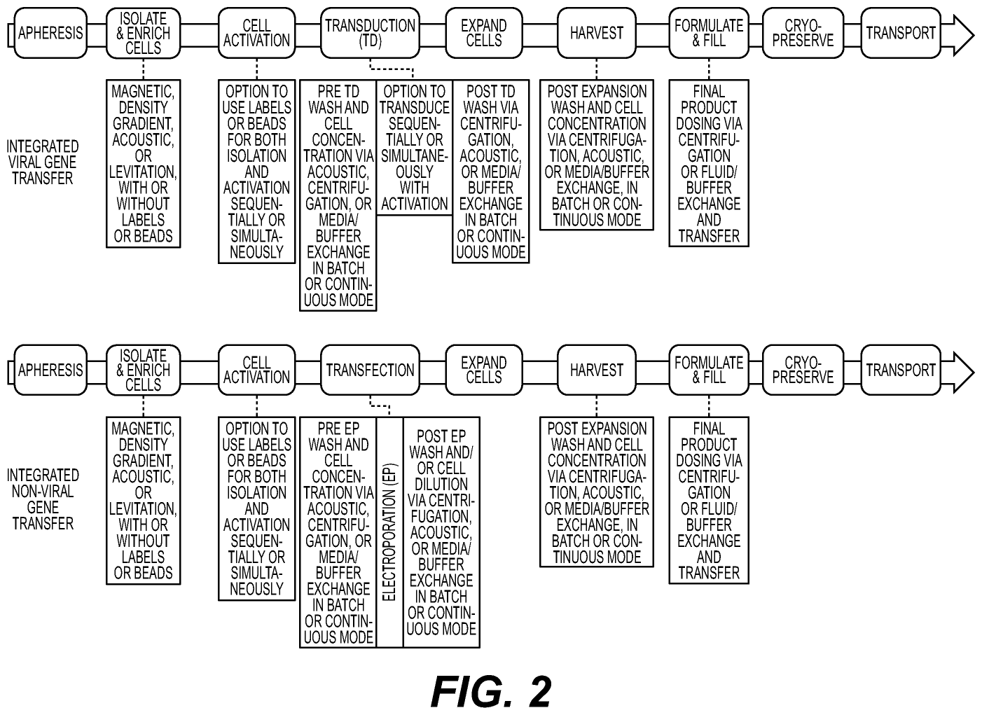

Systems for the process flow embodiments described above are shown in FIG. 2 for both integrated viral gene transfer and for integrated non-viral gene transfer. As shown in FIG. 2 for integrated viral gene transfer, the selection or isolation and enrichment step is performed in an acoustic separation device. In one embodiment, the acoustic separation device may include a single channel single standing wave acoustophoretic blood cell separation system operating in the medium band frequency range that separates lymphocytes from other apheresis blood components based on target cell size, density, and compressibility.

As shown in FIG. 2, the activation step includes the option to use label for both isolation (selection) and activation. The activation step may occur in an output bag from the acoustic separation device or another vessel. During the transduction step as shown in FIG. 2, the cells of interest are placed into a buffer exchange module, transferred to a microfluidic transduction device (MTD), and then back to the buffer exchange module. The cells of interest are then expanded in a closed vessel and then transferred to the acoustic separation device for the harvest step. For formulation and fill, the cells of interest are transferred to the buffer exchange and then the cells of interest are cryopreserved and stored. In certain embodiments, the cells of interest may be transferred between the devices shown in FIG. 2 by traditional manual methods such as gravitational, or pump. In other embodiments, the cells of interest may be transferred between the devices shown in FIG. 2 by a closed system transfer device using syringe fluid handling techniques or via automated controllers (pump+scale, vacuum+light sensor, etc.). Also, it is possible that the cells of interest may be in a culture bag, closed vessel, or may be transferred directly between device through continuous tubing.

As shown in the embodiments of FIG. 2, the integrated non-viral gene transfer embodiment is similar to the integrated viral gene transfer system except that during the transduction or transfection step, an electroporation (EP) step to introduce non-viral gene delivery vehicles used instead of the membrane-based transduction device (MTD) to physically co-localize viral vectors and target cells.

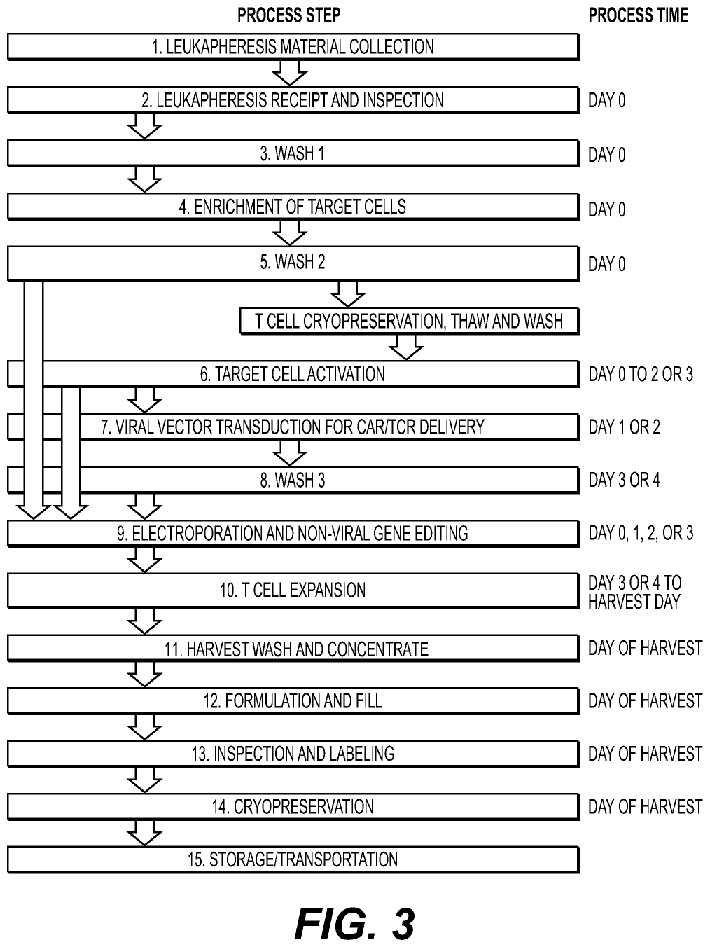

Referring now to FIG. 3 is shown an alternative embodiment of a process flow involving transfection prior to transduction. In this embodiment, step 1 is apheresis or Leukapheresis where a patient's cells are collected at a clinical site, and then shipped to a manufacturing site where the cell therapy will be performed. The incoming leukapheresis material is removed from exterior packaging and inspected at the manufacturing site at step 2. Next, the cells of interest undergo a pre-process washing of the leukapheresis material to reduce volume in step 3. In step 4, the targeted cells are enriched and separated. This includes density gradient enrichment (i.e. PBMC) and conjugated antibody paramagnetic separation. The separation may occur through positive enrichment of T cells (i.e. CD3, TCR-alpha/beta, TCR-gamma/delta, Tscm, Tnaive, Tcm, CD4/CD8 based enrichment) or negative depletion for T cells (i.e. CD19/CD14 based depletion). In this embodiment, the method includes conjugated antibody acoustic separation using positive enrichment of T cells (i.e. CD3, TCR-alpha/beta, TCR-gamma/delta, Tscm, Tnaive, Tcm, CD4/CD8 based enrichment) or negative depletion for T cells (i.e. CD19/CD14 based depletion).

Still referring to FIG. 3, step 5 is a second wash step where the cells of interest are washed and concentrated into growth culture media. After the second wash, excess enriched targeted cells may optionally be cryopreserved. Moving to activation in step 6, activation of the cells of interest may occur in anti-CD3 antibody coated bags in growth culture medium with anti-CD28. In other embodiments, target cell activation using antibody-coated vessels, spheres or particles, and tetrameric antibodies may occur.

Transduction step 7 may include in situ transduction using lentiviral vectors in activation vessel with growth culture medium without enhancing reagents. In another embodiment, transduction may include using lentiviral or retroviral vectors with physical co-localization apparatus with or without enhancing reagents. A third wash at step 8 may occur, where the cells are washed to reduce process impurities. Furthermore, post-wash dilution and inoculation for cell expansion or electroporation/gene editing may occur. Electroporation and non-viral gene editing may occur at step 9. In this step, non-viral plasmid DNA and mRNA are added for gene editing and the cells are electroporated. The cells are then expanded in step 10, where expansion in a growth culture medium may occur in static culture bags, wave-based vessel, or stir tank bioreactors.

Next in step 11 (Harvest wash and concentration), the cells are washed to reduce process impurities and then buffer exchange into cryoprotectant containing buffer at a range of ratio. In step 12 (formulation and fill), the product cells are adjusted with formulation buffer to achieve defined cell densities and once in final formulation are filled into final product bags and vials. The bags and vials will undergo a quality inspection and release test, and will be labeled in step 13. The bags and vials with the product cells will be cryopreserved using controlled-rate freezer (step 14) and then stored and then transported to the clinical site where the patient will receive the engineered cells (step 15).

### CSTD

Throughout the various embodiments described herein, closed-system transfer devices (CSTDs), are used to manually remove and transfer cells in a sterile manner without Grade B/ISO7/BSC environments. In certain embodiments, CSTDs may be used for inline or ex vivo cell sampling, cell and fluid transfer, air addition or air removal, line clearing of process tubing or disposable kit tubing, or to measure fluid volume.

CSTDs may be used to execute these operations in a reduced clean room environment such as Grade C or outside of a BSC. The process flow steps are designed to be compatible with a range of CSTD designs and sizes and enables the execution of closed-system processing between reagent bags, buffer bags, cell culture bags, bags or bottles or vessels exclusive of or inclusive of supporting processing equipment.

In one embodiment, the acoustic cell separation module, transduction module, electroporation module, and buffer exchange module used in the exemplary process flows are compatible with the CSTDs described here. Commercial options of a CSTD include various types of syringes and the Cytiva CPAK-100 and CPAK-101, Millipore NovaSeptum, and devices from EquaShield, all of which are either volume limited or do not perform all the manual closed-system unit operations needed (i.e, volume transfer, cell transfer, air removal, air addition, volume measurement).

In certain embodiments, the CSTDs used in the closed system described herein should maintain functionally-closed system sterility and be able to perform a sampling event on a process vessel in a grade C environment. CSTD is a functionally-closed system that keeps the contents of CSTD and parent vessel sterile and the connection between CSTD and parent vessel also remains sterile. The CSTD in one embodiment is compatible with various cell types, disposable kits, and reagents (i.e. DMSO, media, and etc.) embodied in the process flows described herein. Also, the CSTD keeps samples drawn homogeneous and representative of parent vessel population or formulation. In the described process steps of the various embodiments, the CSTD and packaging should remain free of particulates, extractables, and leechables. In certain embodiments the CSTD should be sterile tube compatible, i.e. Terumo BCT TSCD-II.

In one embodiment, the CSTD may be used for sampling by withdrawing a sample from a parent vessel then transfer sample for analysis. In one example, sampling using the CSTD for post PBMC enrichment (i.e. PremierCell). Also, the CSTD device may be used for cell transfer by withdrawing a sample from a parent vessel then transfer sample into a new parent vessel. In one example, the CSTD can be used to transfer cells from the activation step to the transduction step, and from the transduction step to the expansion seeding step, and then to post wash output bags, or the like.

In other embodiments, the CSTD may be used removing excess air from a parent vessel. In one example, excess air may be removed from an activation bag, a final product formulation bag, or the like. In another embodiment, the CSTD may be used to add air into a parent vessel. In one example, air may be added to a vessel using the CSTD to enable efficient draining of an antibody (e.g., aCD3) buffer or transduction enhancer (e.g., Retronectin) buffer post coat incubation. In addition, the CSTD may be used for volume measurement. In one example, the CSTD may measure the total volume of a parent vessel, such as measuring the volume of harvest wash final output bag.

### Allogeneic CAR-T/TCR System and Method

A system and method for creating allogeneic CAR-T therapy is also described herein. Allogeneic CAR-T therapy is an alternative processing strategy to overcome the inherent limitations of autologous therapy and provide an ‘off-the-shelf’ approach for clinical and commercial product. Allogeneic methods employ T-cells from healthy donors which subsequently undergo gene modifications to confer specificity against tumor antigens. The T-cells may be engineered via gene editing to prevent graft-versus-host disease (GVHD) and the elimination of the allogeneic CAR T cells by the patient's immune system, known as host vs graft rejection.

### Cell Selection

In one embodiment of the allogeneic process, large-scale CD4+/CD8+ T-cell enrichment is carried out via magnetic bead or acoustic selection isolation as described above in the Autologous section with a T-cell recovery of 30-80% (relative to incoming apheresis T-cell composition) at a T-cell purity of more than 90% and viability of typically above 90%. The activation potential of the selected T-cells is targeted, typically, to be between 1.3 to 2.6 growth fold from day 0 to day 3.

### Activation and Buffer Exchange

In one embodiment, the enriched T cells are activated by stimulation of the CD3 or CD3/CD28 or CD2/CD3/CD28 receptors in the presence of IL-2 for 24 to 96 hours. Activated cells are washed via centrifugation or buffer exchanged to concentrate the cells between 15-100 e6 cells/mL in media or electroporation buffer. The buffer exchange concentration rates are designed to enable efficient target throughput levels.

Prior to electroporation in one embodiment, the cells should be exchanged into a medium conducive to the operation. The exchange may be performed by a buffer exchange system that can be operated in batch or continuous mode depending on the type of technology deployed to perform the buffer exchange, i.e. membrane filtration, centrifugation, and microfluidic channels.

Examples of technologies commercially available for buffer exchange are Ekko™ by FloDesgn Sonic (now part of Millipore Sigma), Sefia by Cytiva, Lovo by Fresenius Kabi, and CTS Rotea Counterflow Centrifugation. In addition, multiple companies such as Milipore Sigma, Pall, Sartorius, Repligen, and Cytiva manufacture various forms of membranes (cassette and hollow fiber) for buffer exchange use. Cassette membranes are composed of several individual sheets of membranes in which the feed stream enters from one side of the cassette, the feed is run parallel to the membrane sheets and applied pressure forces the permeable component of the stream to pass through the membrane and the retentate component exists through the designated outlet flow path. Hollow fiber membranes are composed of various amounts of individual cylindrical membranes (fibers) packed side by side in which the feed stream enter from one end of the device into the individual fibers, applied pressure forces the permeable component of the stream to pass through the fibers and the retentate component are retained inside the fiber and exit at the opposite end.

In one embodiment, the buffer exchange operation can process ≥2.5 E9 cells/hr and 0.1 to 10 E9 cells per lot. In addition, the buffer exchange efficiency will be ≥90% and obtain a cell viability ≥90% along with a cell recovery of ≥85%. For buffer exchange using microfluidic channel technology, various systems can be devised for focusing particles suspended within a moving fluid into one or more localized stream lines. The fluid, the channel(s), and the pumping components are configured to cause inertial forces to act on the particles and to focus the particles into one or more stream lines.

### Electroporation and Transduction

In one embodiment, the concentrated cells (15-300 e6/mL) are transfected with genetic or non-genetic material (e.g. DNA or RNA encoding ZFN or CRISPR or TALENs) as described above to affect the desired gene modifications (gene knockout or additions). Post-electroporation, the cells are washed, buffer exchanged, or diluted to minimize exposure to the electroporation buffer during transduction.

In one embodiment, the post-electroporated cells are transduced with construct-encoding lentiviral vectors (LVV) or retroviral vectors (RVV) using enhancing reagents at optimized conditions (retronectin, protamine sulfate, polybrene, or vectrofu sin-1) or enhancer-free physical co-localization viral vector-based gene delivery methods at a cell to vector ratio designed to achieve desired transduction efficiencies and genomic integration. The volume of viral vector is controlled at a target multiplicity of infection (transducing viral particle units per cell) and incorporated into the transduction system or the culture system. The transduction seed density is typically between 1-5 e6 cells/mL (to achieve the desired particle per cell unit ratio) and may last from 1 hour to 72 hours at temperature ranges from 15° C. to 37° C.

### Expansion

In one embodiment, following gene editing and transduction, the cells are expanded in static, shake flasks, rocking wave bioreactors, or stirred tank bioreactors to achieve the desired dose.

### Depletion

In one embodiment, after expansion, the expanded cells are washed via centrifugation, buffer exchange, or acoustic separation to achieve a desired cell concentration of 50-300 e9 cells/mL in 200-500 mL of media. The concentration rate is designed to maintain throughput targets across the unit operations. Depletion may then be performed via a negative selection stepwise isolation step to deplete the unedited TCRab+ cells for improved product purity and quality.

### Cryopreservation

In certain embodiments, after depletion the cells are washed via centrifugation, buffer exchange, or acoustic separation and resuspended in cryopreservation media, dispensed into appropriate bags or vials per dose requirements, and transferred into long term LN2 vapor phase storage.

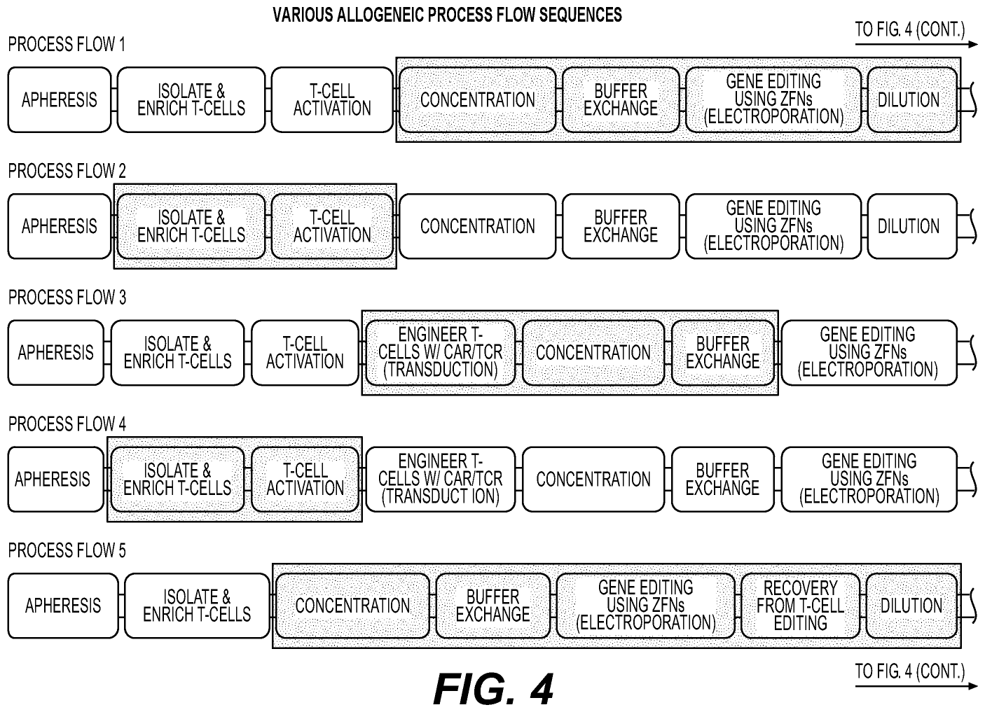

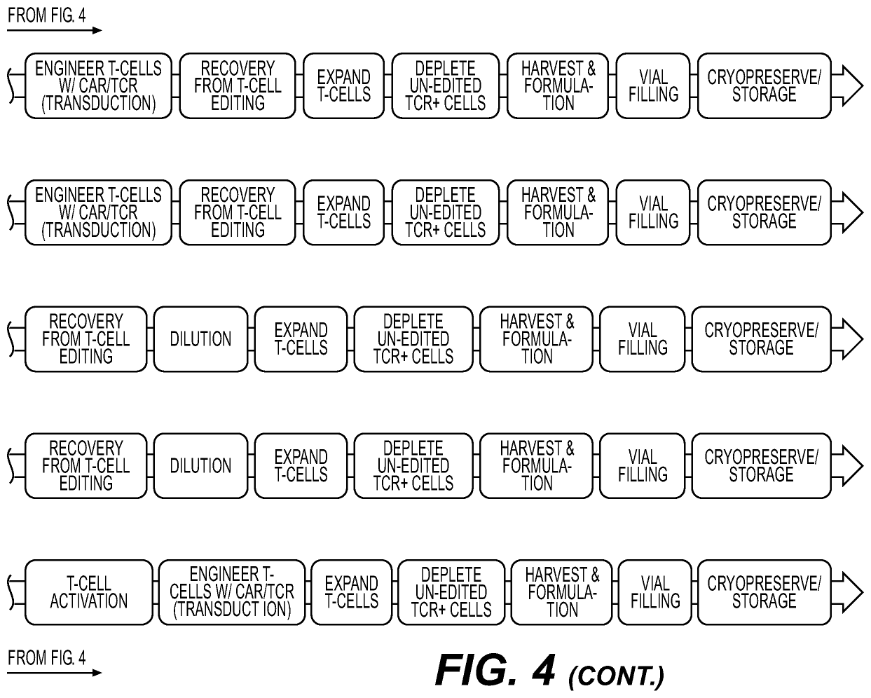

Various embodiments of the allogeneic electroporation and transduction process flows are described in FIG. 4. Each process flow embodiment shown in FIG. 4 may be used for both RVV and LVV processes. Exemplary process flow 1 includes apheresis, selection or isolation and T Cell enrichment followed by T-Cell activation. Next in this process flow, the targeted cells are concentrated in electroporation buffer using a buffer exchange module. After the buffer exchange module, the targeted cells are gene edited using ZFNs (or TALENs, CRISPR/Cas9 or any other nucleases) (Electroporation) and then diluted. Transduction occurs next and then the T-cells recover from T-cell editing and are then expanded in a closed vessel. Following expansion, the cells are depleted to remove un-edited TCR cells. After depletion the targeted cells undergo a harvest was and then are formulated and filled into vials and bags before being cryopreserved.

The following are unique to the individual process flows. For example, process flow 2 is similar to process flow 1 except that process flow 2 combines the T-cell isolation and activation step into one-unit operation. The exemplary process flow 3 is similar to process flow 1 except that the embodiment performs the transduction step prior to the transfection step. Exemplary process flow 4 is similar to process flow 3 except that the embodiment combines the T-cell isolation and activation step into one-unit operation. Also, exemplary process flow 5 is similar to process flow 1 except that the embodiment performs the transfection step prior to activation.

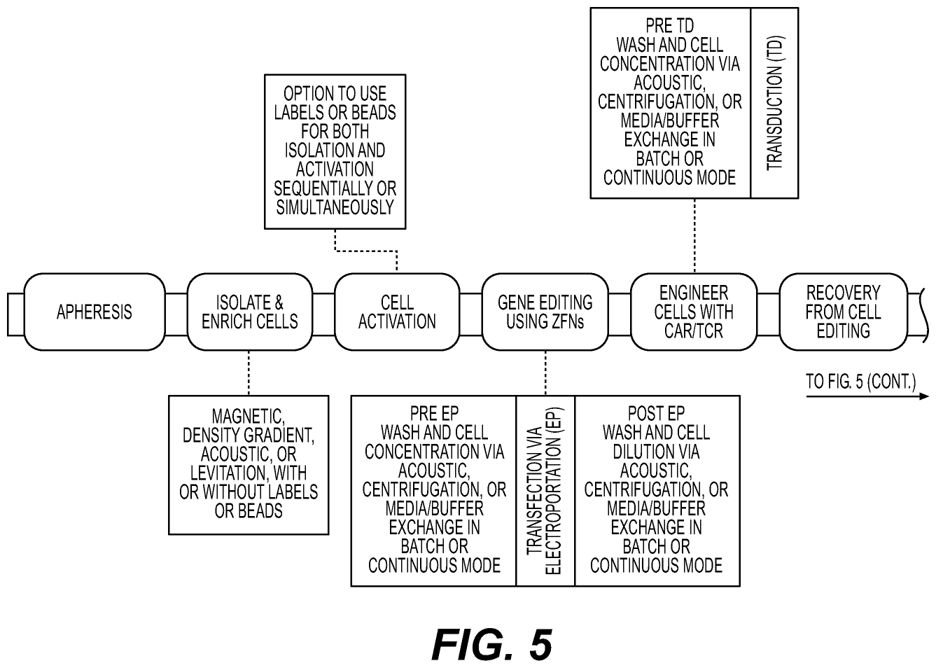

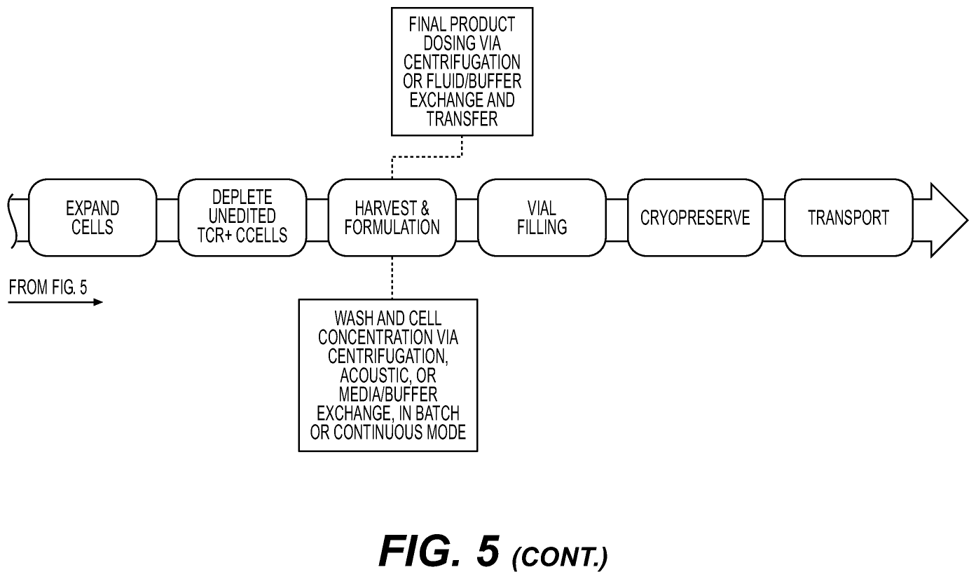

Systems for the process flow embodiments described above for the allogeneic process are shown in FIG. 5. As shown in FIG. 5, selection or isolation and enrichment may occur in an acoustic separation device, and then the isolated cells are activated in a cell bag or other closed vessel. The active cells undergo transfection or gene editing using ZFNs as they are transferred to a buffer exchange module, electroporation (EP), and then back to the buffer exchange module. In the fourth step, the cells are transduced in the buffer exchange module and membrane transduction device (MTD). After recovering from T-Cell editing, the cells are expanded, then un-edited TCR+ cells are depleted. In an acoustic separation device and buffer exchange device, the cells undergo a Harvest wash and are formulated and filled into bags or vials. The bags or vials are then cryopreserved.

### Example 1

In a control process flow as depicted in FIG. 6, the CD4 and CD8 positive T cells are enriched using antibody-conjugated paramagnetic microbeads. The enriched T cells are activated with soluble activator TransAct microbeads for 3 days at 37° C./5% CO2. The activated T cells are transduced with CAR-encoded lentiviral vector by adding the vector directly to the culture vessel with multiplicity of infection (MOI) of 6.8 transducing units per cell. The T cells are further expanded in culture vessel until day 7.

For improved manufacturing method #1, as depicted in FIG. 7, the CD4 and CD8 positive T cells are enriched using antibody-conjugated paramagnetic microbeads. The enriched T cells are activated on anti-CD3 antibody coated culture vessel with soluble anti-CD28 antibody for 24 hours. The activated T cells are transduced with CAR-encoded lentiviral vector by adding the vector directly to the culture vessel at MOI of 4 transducing units per cell following 24 hr activation. The T cells are further expanded in culture vessel until day 7.

For improved manufactured method #2, as depicted in FIG. 8, the CD4 and CD8 positive T cells are enriched using antibody-conjugated paramagnetic microbeads. The enriched T cells are activated on anti-CD3 antibody coated culture vessel with soluble anti-CD28 antibody for 24 hours at 37° C./5% CO2. The activated T cells are transduced with CAR-encoded lentiviral vector by using fluidic membrane-based device to facilitate the co-localizing binding of T cells and vector at MOI of 4 transducing units per cell following 24 hr activation. After the fluidic transduction, T cells are expanded in a culture vessel for cell expansion until day 7.

As shown in Table 1, improved method #1 (FIG. 7) and improved method #2 (FIG. 8) initiated viral transduction following 24 hours of T cell activation on anti-CD3 antibody coated surface, compared to 72 hours of activation using soluble T cell activator. The fluidic viral transduction supported the transduction duration in 4 hours, while the static in situ viral transduction takes 24 to 28 hours in the control method and in improved method #1. From the data produced from 2 healthy donor derived materials (N=2), the T cells showed a similar fold expansion during cell expansion phase. Less MOI (4 transducing units per cell) was used in improved method #1 and improved method #2 than the MOI of 6.8 in control method. Yet the fluidic transduction of improved method #2 and static transduction of improved method #1 based on antibody coated surface activation demonstrated higher CAR expression at 64% and 35%, respectively, compared to the control method.

TABLE 1
Process parameters and manufacturing data of control manufacturing method (FIG.
6) using soluble T cell activator, improved manufacturing method #1 (FIG.
7) (using anti-CD3 antibody coated surface and static lentiviral vector transduction),
and improved manufacturing method #2 (FIG. 8) (using anti-CD3 antibody coated
surface and fluidic co-localizing lentiviral vector transduction)

| | Control | Improved | Improved |
|---|---|---|---|
| Process Parameters and | manufacturing | manufacturing | manufacturing |
| Manufacturing Data | method | method #1 | method #2 |
| Activation method | Soluble activator | Anti-CD3 antibody | Anti-CD3 antibody |
| | (TransAct) | coated surface | coated surface |
| Activation duration | 72 hrs | 24 hrs | 24 hrs |
| Transduction method | Add viral vector to | Add viral vector to the | Fluidic co-localizing |
| | the culture (static) | culture (static) | transduction |
| Transduction duration | 24 to 48 hrs | 24 to 48 hrs | 4 hrs |
| Cell fold expansion (day | 9.84 | 8.01 | 8.84 |
| 7/day 3) N = 2 | | | |
| Multiplicity of Infection | 6.8 transducing units | 4 transducing units per | 4 transducing units |
| for lentiviral vector | per cell | cell | per cell |
| Transduction efficiency | 25% | 35% | 64% |
| (% CAR+ of CD3+) N = 2 | | | |

As an allogeneic CAR-T example for an improved manufacturing method #3 (FIG. 9), the antibody-conjugated magnetic enriched T cells are activated with surface coated anti-CD3 antibody and soluble anti-CD28 antibody. The T cells are transduced in situ with CAR-encoded lentiviral vector in the culture vessel following 1 day of activation. The T cells are electroporated with gene editing mRNA cargos to knock out two genes of interest following 3 days of activation. The transduced and electroporated T cells are recovered overnight at 30° C./5% CO2, then the cells are further expanded in enclosed culture vessel at 37° C./5% CO2 until day 7. The cell growth kinetics, viability, CAR transduction and target gene knockout efficiency were evaluated.

The small-scale and large-scale manufacturing processes representing method #3 are summarized in Table 2, 3 and 4. Throughout the process, the T cells from the small-scale process was able to proliferate from 1.22e7 after electroporation on day 3 to 5.24e8 on day 7 and the T cells in large-scale process could grow from 5.09e8 after electroporation on day 3 to 2.11e9 on day 7. Both small scale and large scale showed consistent and comparable cell viability in the manufacturing process. Similar CAR expression following viral transduction and gene editing were demonstrated in both small-scale and large-scale processes.

TABLE 2
Cell growth for small-scale and large-scale processes
for the improved manufacturing method #3 (FIG. 9)

| | Day 3 post- | | | |
|---|---|---|---|---|
| Cell growth | electroporation | Day 4 | Day 5 | Day 7 |
| Small-scale | 1.22E+07 | 6.62E+07 | 1.40E+08 | 5.24E+08 |
| process | | | | |
| Large-scale | 5.09E+08 | 4.63E+08 | 7.79E+08 | 2.11E+09 |
| process | | | | |

TABLE 3
Cell viability for small-scale and large-scale processes
for the improved manufacturing method #3 (FIG. 9)

| | Day 3 post- | | | |
|---|---|---|---|---|
| Viability | electroporation | Day 4 | Day 5 | Day 7 |
| Small-scale process | 87.6% | 97.7% | 96.1% | 97.4% |
| Large-scale process | 86.9% | 94.4% | 93.8% | 96.1% |

TABLE 4
CAR transduction efficiency and the residual expression
of two target knockout genes on day 7 for the
improved manufacturing method #3 (FIG. 9)

| | | % Target#1 | % Target#2 |
|---|---|---|---|
| | % CAR+ | after KO | after KO |
| Small-scale process | 81.2% | 5.0% | 14.2% |
| Large-scale process | 77.3% | 5.7% | 13.2% |

### Example 2

Dual CAR T cell product are manufactured using improved method #1 (FIG. 7) and method #4 (FIG. 10). In this example, frozen T cells were used as starting material to test a dual CAR construct in the improved method #1 (FIG. 7)-lentiviral vector transduction in large-scale culture and improved method #4 (FIG. 10)-non-viral delivery using large-scale electroporator. In the end of regular 8-day manufacturing, viability of T cells from both methods can reach ≥90% (Table 5). With CD3/CD28 activation, T cells start to proliferate. CAR T cells in improved method #4 were activated one day later than in improved method #1. Proliferation would be expected to be delayed due to later activation. In summary, CAR T cells proliferate well after activation in both methods when compared to their coordinate non-transduced cells control. In the improved method #4, electroporated T cells were able to catch the cell growth in the end of manufacturing compared to their non-transduced cell control (Table 6). The dual CAR T cells from improved method #1 have ˜60% CAR1 expression and ˜40% CAR2 expression. The cells from improved method #4 have ˜30% CAR1 expression and ˜20% CAR2 expression (Table 7). Two donors were tested in both methods and have comparable results, suggesting the robustness of both methods.

TABLE 5
Dual CAR viability from two healthy donors using improved method
#1 (FIG. 7; Day 0 Activation; Day 1 viral transduction)
and method #4 (FIG. 10; Day 0 Electroporation; Day 1 Activation)
Improved method #1

| Days of | Donor 1- | Donor 1- | Donor 2- | Donor 2- |
|---|---|---|---|---|
| manufacturing | NTD | Dual CAR | NTD | Dual CAR |
| 0 | 91 | 91 | 98.3 | 98.3 |
| 1 | 62 | 62 | 52 | 52 |
| 3 | 88 | 87 | 85 | 86 |
| 4 | 90 | 89 | 85 | 85 |
| 5 | 94 | 95 | 91 | 92 |
| 6 | 97 | 96 | 99 | 96 |
| 7 | 97 | 96 | 99 | 99 |
| 8 | 96 | 94 | 96 | 95 |

Improved method #4

| Days post- | Donor 1- | Donor 1- | Donor 2- | Donor 2- |
|---|---|---|---|---|
| Electroporation | NTD | Dual CAR | NTD | Dual CAR |
| 0 | 91 | 91 | 98.3 | 98.3 |
| 1 | 61 | 39 | 67 | 38 |
| 4 | 87 | 63 | 83 | 54 |
| 6 | 94 | 92 | 91 | 90 |
| 8 | 93 | 93 | 92 | 93 |

TABLE 6
Dual CAR T cell fold expansion from two healthy donors
using improved method #1 (FIG. 7; Day 0 Activation;
Day 1 viral transduction) and method #4 (FIG.
10; Day 0 Electroporation; Day 1 Activation)
Improved method #1

| Days of | Donor 1- | Donor 1- | Donor 2- | Donor 2- |
|---|---|---|---|---|
| manufacturing | NTD | Dual CAR | NTD | Dual CAR |
| Activation→0 | 1 | 1 | 1 | 1 |
| 3 | 1.9 | 1.7 | 1.5 | 1.5 |
| 4 | 4.6 | 4.1 | 3.6 | 3.7 |
| 5 | 13.9 | 10.6 | 9.7 | 9.1 |
| 6 | 27 | 36.2 | 33.8 | 37 |
| 7 | 54.1 | 70.1 | 67.7 | 101.9 |
| 8 | 104.7 | 147.4 | 132.5 | 237 |

Improved method #4

| Days post- | Donor 1- | Donor 1- | Donor 2- | Donor 2- |
|---|---|---|---|---|
| Electroporation | NTD | Dual CAR | NTD | Dual CAR |
| Activation→1 | 1 | 1 | 1 | 1 |
| 4 | 4.3 | 0.7 | 2.7 | 1.3 |
| 6 | 12.8 | 11.4 | 6.6 | 11.7 |
| 8 | 70.8 | 55.2 | 40.1 | 59.3 |

TABLE 7
Percentage of CAR1 and CAR2 in T cells from two healthy donors using
improved method #1 (FIG. 7; Day 0 Activation; Day 1 viral transduction)
and method #4 (FIG. 10; Day 0 Electroporation; Day 1 Activation)
Improved method #1

| | CAR1 | | | | CAR 2 | | | |
|---|---|---|---|---|---|---|---|---|
| | | Donor | | Donor | | Donor | | Donor |
| Days of | Donor | 1-Dual | Donor | 2-Dual | Donor | 1-Dual | Donor | 2-Dual |
| manufacturing | 1-NTD | CAR | 2-NTD | CAR | 1-NTD | CAR | 2-NTD | CAR |
| 1 | 0 | 0 | 0 | 0 | 0 | 0 | 0 | 0 |
| 3 | 0 | 52.2 | 0 | 50.3 | 0.12 | 36.39 | 0.02 | 38.98 |
| 4 | 0.89 | 69.2 | 0.03 | 63.8 | 4.79 | 55.34 | 3.79 | 53.35 |
| 8 | 0 | 64.7 | 0.01 | 60.6 | 0.18 | 44.32 | 0.3 | 41.98 |

Improved method #4

| | CAR1 | | | | CAR 2 | | | |
|---|---|---|---|---|---|---|---|---|
| | | Donor | | Donor | | Donor | | Donor |
| Days post- | Donor | 1-Dual | Donor | 2-Dual | Donor | 1-Dual | Donor | 2-Dual |
| electroporation | 1-NTD | CAR | 2-NTD | CAR | 1-NTD | CAR | 2-NTD | CAR |
| 1 | 0.01 | 0.58 | 0.01 | 0.29 | 0.28 | 0.29 | 0.93 | 0.4 |
| 4 | 0.04 | 28.33 | 0.03 | 22.03 | 3.55 | 22.65 | 2.88 | 19.31 |
| 8 | 0 | 32 | 0 | 32.02 | 0.33 | 22.06 | 0.41 | 23.4 |

One skilled in the art will realize the subject matter may be embodied in other specific forms without departing from the spirit or essential characteristics thereof. The foregoing embodiments are therefore to be considered in all respects illustrative rather than limiting of the subject matter described herein.

1. A method for manufacturing CAR expressing human lymphocytes, comprising:
(a) isolating target cells from donor sourced starting material using an isolation technique selected from the group consisting of acoustic separation, antibody-conjugated magnetic beads, density gradient separation, magnetic levitation, antibody conjugated labels, microspheres and any combination thereof;
(b) optionally, contacting the target cells with an activating molecule;
(c) transducing the target cells with CAR construct-encoded lentiviral vectors, retroviral vectors or adeno-associated vectors in an enclosed vessel, a fluidic channel and any combination thereof; and
(d) transfecting the target cells with viral or non-viral genetic material using an electroporation device,
wherein steps (a), (b), (c) and (d) are performed sequentially, in any order, or one or more of steps (a), (b), (c) and (d) are performed simultaneously with the remaining steps performed sequentially in any order.

2. The method of claim 1, wherein the isolation technique of step (a) is acoustic separation, further wherein the purity of the target cells is increased in comparison to isolation of target cells with density, gradient and/or magnetic bead separation.

3. The method of claim 1, wherein the isolation technique of step (a) comprises both acoustic separation and antibody conjugated labels, further wherein the purity of the target cells is increased in comparison to isolation of target cells with density, gradient and/or magnetic bead separation.

4. The method of claim 2, wherein the increased purity of the target cells results at least in part from a reduced presence of monocytes among the target cells.

5. The method of claim 1, wherein after step (d) the target cells are expanded.

6. The method of claim 1, wherein after step (d) the target cells are cryopreserved.

7. (canceled)

8. The method of claim 1, wherein the donor sourced material is selected from the group consisting of previously cryopreserved cells, leukapheresis product, peripheral whole blood, cord blood or any combination thereof.

9. The method of claim 1, wherein the target cells are transduced in a fluidic channel, wherein a fluid flow is provided to co-localizing binding of viral vector and target cells.

10. The method of claim 1, wherein the target cells comprise markers for CD3, CD4, CD8, CD14, CD19, CD25, CD27, CD28, CD34, CD56, CD69, CD95, CCR7, CD62L, CD45RA/RO, PD1, OX40, ICOS and any combination thereof.

11. The method of claim 1, wherein the target cells are isolated using antibody-conjugated magnetic beads wherein one or more antibodies have specificity for a marker selected from the group consisting of CD3, CD4, CD8, CD14, CD19, CD25, CD27, CD28, CD34, CD56, CD69, CD95, CCR7, CD62L, CD45RA/RO, PD1, OX40, ICOS and any combination thereof.

12. The method claim 1, wherein the target cells are isolated using antibody-conjugated beads that respond to an acoustic field wherein one or more antibodies have specificity for a marker selected from the group consisting of CD3, CD4, CD8, CD14, CD19, CD25, CD27, CD28, CD34, CD56, CD69, CD95, CCR7, CD62L, CD45RA/RO, PD1, OX40, ICOS and any combination thereof.

13. The method of claim 1, wherein the target cells are isolated using antibody-conjugated beads that respond to a gravitational field and/or a centrifugation force wherein one or more antibodies have specificity for a marker selected from the group consisting of CD3, CD4, CD8, CD14, CD19, CD25, CD27, CD28, CD34, CD56, CD69, CD95, CCR7, CD62L, CD45RA/RO, PD1, OX40, ICOS and any combination thereof.

14. The method of claim 1, wherein the target cells are activated by contact with a soluble activating reagent selected from the group consisting of MACS® GMP T Cell TransAct™, CD137L, ImmunoCult™ Human CD3 Cell Activator, anti-CD28 antibody, anti-CD3 antibody, Interleukin-2, Interleukin-7, Interleukin-15, Interleukin-3, Interleukin-21, Thermogenesis X-Bacs and any combination thereof or wherein the target cells are activated by contact with an insoluble activating reagent selected from the group consisting of Dynabeads™ Human T-Activator CD3/CD28, Cloudz Human T Cell Activation CD3/CD28 microspheres, CLOUDZ NK Cell Activation CD2/NKp46 microspheres, a microcarrier and any combination thereof.

15. (canceled)

16. The method of claim 1, wherein the target cells are activated in the absence of exogenous IL-2.

17. (canceled)

18. The method of claim 11, wherein the target cells are isolated with two or more antibody-conjugated magnetic beads, wherein each antibody-conjugated magnetic bead has specificity for a different target and the two or more antibody-conjugated magnetic beads are utilized sequentially for target cell selection.

19. The method of claim 1, wherein the target cells are isolated with one or more antibody-conjugated label, wherein each antibody-conjugated label has specificity for a different target and the one or more antibody-conjugated labels are utilized sequentially for target cell selection.

20-21. (canceled)

22. The method of claim 19, wherein the antibody has specificity for CD4 or CD8.

23. (canceled)

24. The method of claim 1, wherein steps (a) and (b) are performed simultaneously.

25. The method of claim 1, wherein steps (a) and (b) are performed simultaneously with anti-CD3 antibody, anti-CD28 antibody, CD137L, Interleukin-7, Interleukin-15, Interleukin-21, and any combination thereof.

26. The method claim 1, wherein the isolation of the target cells in step (a) is performed under a condition selected from the group consisting of a static condition, a circulating condition, a mixing condition, a rocking condition, a suspension condition, a pressurized condition, a laminar flow condition, a turbulent flow condition and any combination thereof.

27. The method of claim 1, wherein the number of target cells is within the range of about 4e7 to about 1e10 cells.

28. (canceled)

29. The method of claim 1, wherein the target cells are transduced in step (c) with viral vector within 0 to 72 hours of activation in step (b), in the presence of an enhancing reagent selected from the group consisting of Retronectin, protamine sulfate, polybrene, LentiBOOST, ViralEntry™, Vectofusin-1 and any combination thereof.

30. (canceled)

31. The method of claim 1, wherein the target cells are transduced in step (c) with viral vector within 0 to 72 hour of activation in step (b), in the absence of an exogenous enhancing reagent.

32. The method of claim 1, wherein the target cells are transduced in a fluidic channel, wherein the fluidic channel is comprised within a fluidic transmembrane device which provides an enclosed system with transmembrane flow and further provides for colocalization of the viral vector and the target cells onto a membrane with a molecular weight cut-off between about 200 kDa and about 1000 kDa.

33. The method of claim 1, wherein the transfection of step (d) precedes the activation of step (b) further wherein the target cells are contacted with plasmid DNA, mRNA, siRNA, or microRNA in step (d).

34. The method of claim 1, wherein the transfection of step (d) follows the contacting with an activating molecule of step (b) further wherein the target cells are contacted with plasmid DNA, mRNA, siRNA, or microRNA in step (d).

35. The method of claim 1, wherein the target cells are transfected with a cargo selected from the group consisting of a zinc finger nuclease mRNA, a TALEN mRNA, a CRISPR guided RNA/Cas ribonucleoprotein, or any combination thereof.

36. The method of claim 1, wherein the electroporation device is an enclosed system that generates pulsed waveforms to electroporate about 1e6 to about 1e10 target cells in batches using semi continuous flow or using continuous flow.

37. (canceled)

38. The method of claim 1, wherein the electric charge pulse for electroporation is a combination of 10 to 100 kV/m, 10 μs to 30 ms, for 1 to 30 pulses.

39. The method of claim 1, wherein the target cells are washed and concentrated into electroporation buffer or culture media using an enclosed centrifugation system to a cell concentration between about 2e7 and 1.5e8 cells per mL.

40. The method of claim 1, wherein the target cells are expanded after transfection using expansion feeding.

41. (canceled)

42. The method of claim 1, wherein the target cells are expanded after transfection without using expansion feeding.

43. The method of claim 1, wherein the activating of step (b) is performed for up to 96 hours at about 37° C. and about 5% CO2.

44. The method of claim 1, further comprising performing a closed-system centrifugation wash to the target cells after the contacting with an activating molecule of step (b).

45. The method of claim 1, wherein the transducing of step (c) is performed with a CAR construct-encoded lentiviral vector, retroviral vector or adeno-associated vector using an enhancing reagent.

46. (canceled)

47. The method of claim 1, wherein the transducing of step (c) is performed with a CAR construct-encoded lentiviral vector, retroviral vector or adeno-associated vector without using an enhancing reagent.

48. The method of claim 1, wherein the transducing of step (c) is performed for 1 to 72 hours at a temperature between 15° C. to 37° C.

49. The method of claim 1, wherein the activating of step (b) is performed sequentially prior to the transducing of step (c).

50. The method of claim 1, wherein the contacting with an activating molecule of step (b) is performed simultaneously with the transducing of step (c).

51-56. (canceled)
